(19) United States
(12) Patent Application Publication
(10) Pub. No.: US 2004/0156828 A1
(43) Pub. Date: Aug. 12, 2004

(54) Adeno-associated virus mediated B7.1 vaccination synergizes with angiostatin to eradicate disseminated liver metastatic cancers

(76) Inventors: Ruian Xu, Hong Kong (CN); Xueying Sun, Auckland (NZ); Sheung-Tat Fan, Hong Kong (CN); Hsiang-Fu Kung, Hong Kong (CN); Geoffrey Krissansen, Auckland (NZ); Peter Chin Wan Fung, Hong Kong (CN)

Correspondence Address: JONES DAY, 222 EAST 41ST ST, NEW YORK, NY, 10017, US

(21) Appl. No.: 10/753,164

(22) Filed: Jan. 7, 2004

Related U.S. Application Data
(60) Provisional application No. 60/438,449, filed on Jan. 7, 2003.

(51) Int. Cl. A61K 48/00; C12N 15/861

(52) U.S. Cl. 424/93.2; 435/456; 435/325; 435/320.1; 536/23.2

(57) ABSTRACT

The present invention provides adeno-associated viral (AAV) vectors encoding an angiostatin protein (“AAV-angiostatin vector”) and/or a costimulatory molecule B7.1 (“AAV-B7.1 vector”). The AAV-angiostatin vector can be administered to a subject, alone or in combination, sequentially or simultaneously, with a AAV-B7.1 vector for treatment, management or prevention of metastatic tumors. Pharmaceutical compositions and vaccines comprising the AAV-angiostatin vector and/or the AAV-B7.1 vector and methods of manufacturing are also described. Administration of AAV-angiostatin and AAV-B7.1 vectors by intraportal and muscular injections are also provided.

The present application claims priority to U.S. Provisional Application Serial No. 60/438,449, filed Jan. 7, 2003, which is incorporated herein by reference in its entirety.

### 1. INTRODUCTION

The present invention relates to a therapeutic agent and methods for preventing, treating, managing, or ameliorating tumors and/or cancers of all types including but not limited to, metastatic liver cancer, using said therapeutic agent. In particular, the present invention provides a nucleic acid molecule comprising an adeno-associated viral (AAV) vector, operably linked to a sequence encoding angiostatin protein and/or costimulatory molecule B7.1. In particular, the present invention relates to an AAV vector encoding a costimulatory molecule B7.1 (“AAV-B7.1 vector”) useful for treating liver metastatic tumors. The AAV-B7.1 vector can be administered to a subject, preferably a human, alone or in combination, sequentially or simultaneously, with a second AAV vector encoding angiostatin (“AAV-angiostatin vector”). The invention also relates to an AAV vector encoding both the costimulatory molecule B7.1 and angiostatin (“AAV-B7.1/angiostatin vector”). Pharmaceutical compositions and vaccines comprising the AAV-B7.1 vector, the AAV-angiostatin vector, and/or the AAV-B7.1/angiostatin vector are encompassed by the present invention. Methods for making and using the AAV vectors, pharmaceutical compositions and vaccines are also described. In particular, the invention is directed to methods of treatment and prevention of cancer by the administration of an effective amount of the AAV-B7.1 vector, the AAV-angiostatin vector, and/or the AAV-B7.1/angiostatin vector. In other embodiments, the methods further provide combination treatment with surgery, standard and experimental chemotherapies, hormonal therapies, biological therapies, immunotherapies, radiation therapies, embolization, and/or chemoembolization therapies for the treatment or prevention of cancer.

### 2. BACKGROUND OF THE INVENTION

2.1 Metastatic Liver Cancer

The liver is the most frequent site of blood-borne metastases, and is involved in about one-third of all cancers, including the most frequent cancer types (Fidler I. J. et al. The implications of angiogenesis for the biology and therapy of cancer metastasis. Cell 1994; 79: 185-8; Weinstat-Saslow D. et al. Angiogenesis and colonization in the tumor metastatic process: basic and applied advances. FASEB J. 1994; 8: 401-7). Metastatic liver cancer has a very poor prognosis and lacks effective therapy. Despite extensive exploration for novel therapies, there is no effective treatment for liver metastases. Most patients die within one year after diagnosis. Chemotherapy and embolization are at best palliative, with no impact on survival or longevity. Resection of liver metastasis constitutes the only curative treatment, but is feasible for only 10% of patients, and the recurrence rate remains very high after tumor resection. There is therefore an urgent need to seek potential therapeutic strategies for the treatment of metastatic liver malignancies.

2.2 Anti-Angiogenesis Therapy

Although numerous endogenous angiogenesis inhibitors have been discovered, the clinical evaluation of these agents has been hindered by high dose requirements, manufacturing constraints, and the relative instability of the corresponding recombinant proteins. Regressed tumors regrew when therapy with angiostatin was suspended. Prolonged tumor dormancy could be achieved by several rounds of therapy (Holmgren L. et al., 1995, supra; O'Reilly M. S. et al., 1996, supra). So far the therapeutic effects of angiostatin remain controversial, partly because the circulating life of the angiostatin is very short and the local concentration of angiostatin is not high enough to meet the therapeutic requirement. Although one study has indicated that the concentration of endostatin, another anti-angiogenesis drug, in circulation after administration of purified protein could reach up to 400 μg/ml (Blezinger P. et al. Systemic inhibition of tumor growth and tumor metastases by intramuscular administration of the endostatin gene. Nature Biotechnol. 1999; 17: 343-348), it is difficult to determine how high the local concentration of such protein is in situ. Therefore, gene therapy in which the angiostatin gene is delivered to tumors and their proximity and expressed stably for a long period of time, has become increasingly attractive.

There is an urgent need for an ideal vector for cancer gene therapy which provide greater efficacy and reduced toxicity over currently available agents.

2.3 Adeno-Associated Virus Expression Vector

Adeno-associated virus (AAV) is a nonpathogenic, helper-dependent member of the parvovirus family with several major advantages such as stable integration, low immunogenicity, long-term expression, and the ability to infect both dividing and nondividing cells.

The present inventors have established a fast and persistent expression system induced by an adeno-associated virus. With this system, it has been previously demonstrated that intraportal injection of AAV expression vector encoding an angiogenic inhibitor led to high-level, long-term (6 months), and persistent transgene expression of angiostatin localized to hepatocytes, and significant suppression of the growth of both nodular and disseminated metastatic EL-4 lymphoma tumors established in the liver (see U.S. Provisional Application No. 60/438,449, filed Jan. 7, 2003; and Xu R. et al. Long-term expression of angiostatin suppresses liver metastatic cancer in mice. Hepatology. 2003; 37(6): 1451-60, which are incorporated herein by reference in their entireties).

2.4 Costimulatory Molecule B7.1

Two major obstacles for achieving a tumor-specific immune response include (1) overcoming peripheral T cell tolerance against tumor self-antigens (Ags), and (2) inducing cytotoxic T lymphocytes (CTLs) that effectively eradicate disseminated tumor metastases and subsequently maintain a long-lasting immunological memory preventing tumor recurrence Induction of tumor-specific CTLs requires at least two signals: (a) tumor antigens that are processed and presented by major histocompatibility complex (MHC) class I and/or class II molecules on the surface of antigen-presenting cells (APCs); and (b) sufficient levels of costimulatory molecules on tumor cells or other APCs (Mueller D. L. et al. Clonal expansion versus functional clonal inactivation: a costimulatory signalling pathway determines the outcome of T cell antigen. Annu Rev Immunol. 1989; 7: 445-80). The B7 family of membrane proteins are the most potent of the costimulatory molecules and interact with CD28 and CTLA-4 on the T cell surface (Galea-Lauri J. et al. Novel costimulators in the immune gene therapy of cancer. Cancer Gene Ther. 1996; 3: 202-14).

Optimized gene transfer of several T cell costimulatory cell adhesion molecules (CAMs) including B7.1 can lead to tumor specific T cell proliferation and cytotoxicity and protective immunity against a parental tumor challenge. However, CAM-mediated immunotherapy is problematical in that it is ineffective against large tumors, and generates weak anti-tumor systemic immunity (Kanwar J. R. et al. Taking lessons from dendritic cells: multiple xenogeneic ligands for leukocyte integrins have the potential to stimulate anti-tumour immunity. Gene Therapy 1999; 6: 1835-1844). Accordingly, a more effective treatment method is urgently needed.

### 3. SUMMARY OF THE INVENTION

The present invention is based, in part, on the observations by the present inventors that novel adeno-associated virus (AAV) vectors lead to persistent (>6 months) expression of a transgene in both gut epithelial cells and hepatocytes, resulting in long-term phenotypic recovery in a diabetic animal model (Xu R. A. et al., Perarolly transduction of diffuse cells and hepatocyte insulin leading to euglycemia in diabetic rats. Mol Ther. 2001; 3: S180; During M. J. et al. Perarolly gene therapy of lactose intolerance using an adeno-associated virus vector. Nature Med. 1998; 4: 1131-1135; During M. J. et al. An oral vaccine against NMDAR1 with efficacy in experimental stroke and epilepsy. Science 2000; 287: 1453-1460).

To overcome the problems in cancer treatments, the present inventors discovered that the immune system can be harnessed as a potent weapon to combat cancer, but only if immunotherapy is combined with treatment strategies that target a tumor's weapons of survival, defense, and attack. If cancer cells are prevented from growing they will be unable to generate immune escape variants. In searching for ways to more effectively harness and strengthen the anti-tumor activity of CAM-mediated immunotherapy, the present inventors have engineered a new recombinant AAV vector encoding the T cell costimulator B7.1. Further, the present inventors have developed a novel immuno-gene therapy for treatment of cancer by administering B7.1 with anti-angiogenic agents such as angiostatin (Sun X. et al. Cancer Gene Ther. 2001; 8: 719-727, which is incorporated herein by reference in its entirety). The present inventors have also developed a novel immuno-gene therapy for cancer by administering angiostatin, B7.1 and/or anti-sense Hypoxia-inducible-factor 1 (Sun X. et al. Gene transfer of antisense hypoxia inducible factor-I enhances the therapeutic efficacy of cancer immunotherapy. Gene Ther. 2001; 8: 638-645, which is incorporated herein by reference in its entirety). This particular combination of reagents has synergistic effects in treating cancer. In particular, the present invention shows that combination therapy overcomes tumor immune-resistance and causes the complete and rapid eradication of large tumor burdens, which are refractory to monotherapy with either angiostatin, or antisense Hypoxia-inducible-factor 1 or B7.1.

Accordingly, the present invention provides a therapeutic agent for preventing, treating, managing, or ameliorating various tumors and/or cancers, including, but not limited to, liver cancers. Specifically, the invention provides a therapeutic agent for treating liver cancer, in particular, disseminated metastatic liver cancer, by way of gene therapy. In a specific embodiment, the therapeutic agent of the present invention comprises a nucleic acid molecule comprising an adeno-associated viral vector, a beta-actin promoter, a cytomegalovirus enhancer, and a woodchuck hepatitis B virus post-transcriptional regulatory element, operably linked to a sequence encoding angiostatin protein and/or costimulatory molecule B7.1. In a specific embodiment, the AAV vector encodes a costimulatory molecule B7.1 (“AAV-B7.1 vector”). In another specific embodiment, the AAV vector encodes angiostatin (“AAV-angiostatin vector”). In yet another specific embodiment, the invention also relates to an AAV vector encoding both the costimulatory molecule B7.1 and angiostatin (“AAV-B7.1/angiostatin vector”). The invention relates to the administration of the AAV-B7.1 vector, alone or in combination, sequentially or simultaneously, with the AAV-angiostatin vector and/or AAV-B7.1/angiostatin vector to a subject, preferably a human. The AAV-B7.1 vector, the AAV-angiostatin vector, and the AAV-B7.1/angiostatin vector are useful for treating or preventing cancer, preferably metastatic tumors, more preferably liver metastatic tumors.

In certain embodiments, the invention relates to nucleic acid molecules comprising an AAV vector. In one embodiment, the nucleic acid molecule comprises an AAV vector and a cytomegalovirus enhancer and beta-actin promoter (CAG promoter) which is operably linked to a nucleic acid sequence encoding angiostatin. In a specific embodiment, the nucleic acid molecule comprises an AAV vector and a CAG promoter which is operably linked to either the nucleotide sequence of SEQ ID NO:1 or a nucleotide sequence that encodes the amino acid sequence of SEQ ID NO:2.

In another embodiment, the nucleic acid molecule comprises an AAV and a CAG promoter which is operably linked to a nucleic acid sequence encoding costimulator B7.1. In a specific embodiment, the nucleic acid molecule comprises an AAV vector and a CAG promoter which is operably linked to either the nucleotide sequence of SEQ ID NO:3 or a nucleotide sequence that encodes the amino acid sequence of SEQ ID NO:4. In another specific embodiment, the nucleic acid molecule comprises an AAV vector and a CAG promoter which is operably linked to either the nucleotide sequence of SEQ ID NO:5 or a nucleotide sequence that encodes the amino acid sequence of SEQ ID NO:6. In specific embodiments, the nucleotide sequence encoding B7.1 that may be used in the present invention include those deposited with GenBank® having accession nos. NM_{—}005191 (SEQ ID NO:3) and X60958 (SEQ ID NO:5). The nucleic acid molecules can further comprise a woodchuck hepatitis B virus post-transcriptional regulatory element (WPRE).

The invention also relates to vectors comprising the nucleic acid molecules described above. In a specific embodiment, said vector is an AAV containing a CAG promoter which is operatively linked to the nucleotide sequence encoding angiostatin. In another specific embodiment, said vector is an AAV vector containing EGR-1 promoter and target specific promoter albumin. In a preferred embodiment, the vector comprises a CAG promoter which is operatively linked to the nucleotide sequence encoding the angiostatin protein having an amino acid sequence of SEQ ID NO:2 or a biologically functional fragment, analog, or variant thereof. In one embodiment, said nucleotide sequence has a nucleotide sequence of SEQ ID NO:1. In another embodiment, said nucleotide sequence has a nucleotide sequence that hybridizes under stringent conditions, as herein defined, to a complement of the nucleotide sequence of SEQ ID NO:1, wherein said nucleotide sequence encodes proteins or polypeptides which exhibit at least one structural and/or functional feature of angiostatin. In yet another embodiment, said nucleotide sequence has a first nucleotide sequence that hybridizes under stringent conditions to a complement of a second nucleotide sequence encoding an amino acid sequence of SEQ ID NO:2 or a fragment thereof, wherein the first nucleotide sequence encodes proteins or polypeptides which exhibit at least one structural and/or functional feature of angiostatin.

In another specific embodiment, said vector is an AAV containing a CAG promoter which is operatively linked to the nucleotide sequence encoding B7.1. In another specific embodiment, said vector is an AAV vector containing EGR-1 promoter and target specific promoter albumin. In a preferred embodiment, the vector comprises a CAG promoter which is operatively linked to the nucleotide sequence encoding the B7.1 protein having an amino acid sequence of SEQ ID NO:4 or 6, or a biologically functional fragment, analog, or variant thereof. In one embodiment, said nucleotide sequence has a nucleotide sequence of SEQ ID NO:3 or 5. In another embodiment, said nucleotide sequence has a nucleotide sequence that hybridizes under stringent conditions, as herein defined, to a complement of the nucleotide sequence of SEQ ID NO:3 or 5, wherein said nucleotide sequence encodes proteins or polypeptides which exhibit at least one structural and/or functional feature of B7.1. In yet another embodiment, said nucleotide sequence has a first nucleotide sequence that hybridizes under stringent conditions to a complement of a second nucleotide sequence encoding an amino acid sequence of SEQ ID NO:4 or 6 or a fragment thereof, wherein the first nucleotide sequence encodes proteins or polypeptides which exhibit at least one structural and/or functional feature of B7.1.

In certain other embodiments, the nucleic acid molecule comprises an AAV vector and a cytomegalovirus enhancer and beta-actin promoter (CAG promoter) which is operably linked to a first nucleic acid sequence encoding angiostatin and a second nucleic acid sequence encoding B7.1. The expression of the second nucleic acid molecule may be driven by a CAG promoter or a different promoter. In a specific embodiment, the nucleic acid molecule comprises an AAV vector and a CAG promoter which is operably linked to a first polynucleotide that comprises the nucleotide sequence of SEQ ID NO:1 or encodes the amino acid sequence of SEQ ID NO:2, and a second polynucleotide sequence that comprises the nucleotide sequence of SEQ ID NO:3 or encodes the amino acid sequence of SEQ ID NO:4. In another specific embodiment, the nucleic acid molecule comprises an AAV vector and a CAG promoter which is operably linked to a first polynucleotide that comprises the nucleotide sequence of SEQ ID NO:1 or encodes the amino acid sequence of SEQ ID NO:2, and a second polynucleotide sequence that comprises the nucleotide sequence of SEQ ID NO:5 or encodes the amino acid sequence of SEQ ID NO:6.

Host cells comprising the vectors are also encompassed by the present invention. The invention further relates to pharmaceutical compositions comprising the nucleic acid molecules and a pharmaceutically acceptable carrier.

In one embodiment, the invention provides methods for isolating and purifying B7.1 protein, or a fragment, variant, or derivative thereof. The invention also provides methods for isolating and purifying angiostatin protein, or a fragment, variant, or derivative thereof.

The invention further relates to methods of treating or preventing cancer in a subject by administering to said subject a therapeutically or prophylactically effective amount of one or more nucleic acid molecules comprising an AAV-B7.1 vector and/or an AAV-angiostatin vector of the present invention. In particular, the present invention provides a combination therapy for treating metastatic tumors comprising administering by intraportal or muscular route to a subject the AAV-B7.1 vector, followed by intraportal or muscular injection of the AAV-angiostatin vector. In another embodiment, the invention relates to method for treating metastatic tumors comprising administering to a subject one or more AAV-B7.1 vectors, AAV-angiostatin vectors, and/or AAV-B7.1/angiostatin vectors. In a specific embodiment, a first AAV-B7.1 vector, AAV-angiostatin vector, and/or AAV-B7.1/angiostatin vector may be administered by intraportal or muscular injection, followed by intraportal or muscular injection of a second AAV-B7.1 vector, AAV-angiostatin vector, and/or AAV-B7.1/angiostatin vector.

In a specific embodiment, the cancer is liver cancer. In a more specific embodiment, the liver cancer is metastatic. The AAV-B7.1 vector, AAV-angiostatin vector, and/or AAV-B7.1/angiostatin vector may be intravenously injected or transfused into the subject, preferably via a portal vein.

The present invention also provides a pharmaceutical composition comprising the therapeutic agent of the present invention and a pharmaceutically acceptable carrier. In addition, the present invention provides methods for preparing pharmaceutical compositions for modulating the expression or activity of the therapeutic agent of the invention. Such methods comprise formulating a pharmaceutically acceptable carrier with an agent which modulates expression or activity of the therapeutic agent of the invention. Such compositions can further include additional active agents. The methods of the present invention further comprise one or more other treatment methods such as surgery, standard and experimental chemotherapies, hormonal therapies, biological therapies, immunotherapies, radiation therapies, embolization, and/or chemoembolization therapies.

Furthermore, the present invention provides a method of preventing, treating, managing, or ameliorating various tumors and/or cancers, including, but not limited to, liver cancers, in a subject, comprising administering to the subject a prophylactically or therapeutically effective amount of the therapeutic agent of the present invention. The tumors and/or cancers may be either primary or metastasized. In one aspect, the therapeutic agent of the present invention is administered to the subject systemically, for example, by intravenous, intramuscular, or subcutaneous injection, or oral administration. In another aspect, the therapeutic agent is administered to the subject locally, for example, by injection to a local blood vessel which supply blood to a particular organ, tissue, or cell afflicted by disorders or diseases, or by spraying or applying suppository onto afflicted areas of the body. In a specific embodiment, the methods of the present invention can be applied to prevent, treat, manage, or ameliorate liver cancer, wherein the therapeutic agent is administered via vein injection, muscles injection, and oral route. In a preferred embodiment, the therapeutic agent is administered locally by intraportal vein injection.

3.1 Definition

As used herein, the term “analog,” especially “angiostatin analog,” refers to any member of a series of peptides or nucleic acid molecules having a common biological activity, including antigenicity/immunogenicity and antiangiogenic activity, and/or structural domain and having sufficient amino acid or nucleotide sequence identity as defined herein. Angiostatin analog can be from either the same or different species of animals. Similarly, B7.1 analog can be from either the same or different species of animals.

As used herein, the term “angiostatin” or “angiostatin protein” refers to an angiostatin protein, fragment, variant or derivative, from any species. Angiostatin may be from primates, including human, or non-primates, including porcine, bovine, mouse, rat, and chicken, etc. One example of angiostatin protein comprises the amino acid sequence of SEQ ID NO:2. Another example of angiostatin protein comprises an amino acid sequence encoded by the nucleotide sequence of SEQ ID NO:1 or a nucleotide sequence that hybridizes under stringent condition to SEQ ID NO:1. Angiostatin also refers to a functionally active angiostatin protein (i.e., having angiostatin activity as assessed by the methods as described infra in Section 6), fragments, derivatives and analogs thereof. Angiostatin useful for the present invention includes angiostatin comprising or consisting of the amino acid sequence of SEQ ID NO:2 or having an amino acid sequence comprising substitutions, deletions, inversions, or insertions of one, two, three, or more amino acid residues, consecutive or non-consecutive, with respect to SEQ ID NO:2 and retaining angiostatin activity; and naturally occurring variants of mouse angiostatin. Particularly useful angiostatin protein is human angiostatin.

As used herein, the term “B7.1” or “B7.1 protein” refers to a B7.1 costimulatory molecule or costimulator protein, fragment, variant or derivative, from any species. B7.1 may be from primates, including human, or non-primates, including porcine, bovine, mouse, rat, and chicken, etc. One example of B7.1 protein comprises the amino acid sequence of SEQ ID NO:4 or 6. Another example of B7.1 protein comprises an amino acid sequence encoded by the nucleotide sequence of SEQ ID NO:3 or 5, or a nucleotide sequence that hybridizes under stringent condition to SEQ ID NO:3 or 5. B7.1 also refers to a functionally active B7.1 protein (i.e., having B7.1 activity as assessed by the methods as described infra in Section 6), fragments, derivatives and analogs thereof. Angiostatin useful for the present invention includes B7.1 comprising or consisting of the amino acid sequence of SEQ ID NO:4 or having an amino acid sequence comprising substitutions, deletions, inversions, or insertions of one, two, three, or more amino acid residues, consecutive or non-consecutive, with respect to SEQ ID NO:4 or 6 and retaining angiostatin activity; and naturally occurring variants of mouse angiostatin. Particularly useful B7.1 protein is mouse and human B7.1.

A “conservative amino acid substitution” is one in which the amino acid residue is replaced with an amino acid residue having a side chain with a similar charge. A “non-conservative amino acid substitution” is one in which the amino acid residue is replaced with an amino acid residue having a side chain with an opposite charge. Families of amino acid residues having side chains with similar charges have been defined in the art. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing

=cysteine and methionine. (See, for example, Biochemistry, 4th ed., Ed. by L. Stryer, WH Freeman and Co. 1995).

As used herein, the term “variant” refers either to a naturally occurring allelic variation of a given peptide or a recombinantly prepared variation of a given peptide or protein in which one or more amino acid residues have been modified by amino acid substitution, addition, or deletion.

As used herein, the term “derivative” refers to a variation of given peptide or protein that are otherwise modified, i.e., by covalent attachment of any type of molecule, preferably having bioactivity, to the peptide or protein, including non-naturally occurring amino acids.

As used herein, the term “fragments” includes a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino acid residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, at least contiguous 250 amino acid residues, at least 300 amino acid residues, at least 350 amino acid residues, at least 400 amino acid residues, at least 450 amino acid residues, at least 500 amino acid residues, at least 550 amino acid residues, at least 600 amino acid residues, at least 650 amino acid residues, at least 700 amino acid residues, at least 750 amino acid residues, at least 800 amino acid residues, at least 850 amino acid residues, at least 900 amino acid residues, or multiples thereof, of the amino acid sequence of a polypeptide, preferably that has angiostatin or B7.1 activity.

As used herein, an “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment of the invention, nucleic acid molecules encoding polypeptides/proteins of the invention are isolated or purified. The term “isolated” nucleic acid molecule does not include a nucleic acid that is a member of a library that has not been purified away from other library clones containing other nucleic acid molecules.

As used herein, the term “in combination” refers to the use of more than one prophylactic and/or therapeutic agents.

As used herein, the terms “manage,” “managing” and “management” refer to the beneficial effects that a subject derives from a prophylactic or therapeutic agent, which do not result in a cure of the disease or disorder. In certain embodiments, a subject is administered one or more prophylactic or therapeutic agents to “manage” a disease or disorder so as to prevent the progression or worsening of the disease or disorder.

As used herein, the terms “prevent,” “preventing” and “prevention” refer to the prevention of the a disease or disorder in a subject resulting from the administration of a prophylactic or therapeutic agent.

As used herein, the term “prophylactically effective amount” refers to that amount of the prophylactic agent sufficient to prevent a disease or disorder associated with a cell population and, preferably, result in the prevention in proliferation of the cells. A prophylactically effective amount may refer to the amount of prophylactic agent sufficient to prevent the proliferation of cells in a patient.

As used herein, the term “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from chemotherapy include, but are not limited to, gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure, constipation, nerve and muscle effects, temporary or permanent damage to kidneys and bladder, flu-like symptoms, fluid retention, and temporary or permanent infertility. Side effects from radiation therapy include but are not limited to fatigue, dry mouth, loss of appetite and hair loss. Other side effects include gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (56^{th} ed., 2002).

As used herein, the term “under stringent condition” refers to hybridization and washing conditions under which nucleotide sequences having at least 70%, at least 75%, at least 80%, at least 85%, at least 90%, or at least 95% identity to each other remain hybridized to each other. Such hybridization conditions are described in, for example but not limited to, Current Protocols in Molecular Biology, John Wiley & Sons, N.Y. (1989), 6.3.1-6.3.6.; Basic Methods in Molecular Biology, Elsevier Science Publishing Co., Inc., N.Y. (1986), pp. 75-78, and 84-87; and Molecular Cloning, Cold Spring Harbor Laboratory, N.Y. (1982), pp. 387-389, and are well known to those skilled in the art. A preferred, non-limiting example of stringent hybridization conditions is hybridization in 6×sodium chloride/sodium citrate (SSC), 0.5% SDS at about 68° C. followed by one or more washes in 2×SSC, 0.5% SDS at room temperature. Another preferred, non-limiting example of stringent hybridization conditions is hybridization in 6×SSC at about 45° C. followed by one or more washes in 0.2×SSC, 0.1% SDS at about 50-65° C. Yet anotherpreferred, non-limiting example of stringent hybridization conditions is to employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or to employ 50% formamide, 5×SSC (0.75 M NaCl, 0.075 M Sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the terms “therapeutic agent” and “therapeutic agents” refer to any agent(s) that can be used in the prevention, treatment, or management of diseases or disorders associated with a cell population. The term “therapeutic agent” refers to a composition comprising one or more vector of the present invention encoding angiostatin or B7.1 protein.

As used herein, the term “therapeutically effective amount” refers to that amount of the therapeutic agent sufficient to treat, manage, or ameliorate a disease or disorder associated with a cell population. A therapeutically effective amount may refer to the amount of therapeutic agent sufficient to reduce the number of cells or to delay or minimize the spread of cells (e.g., reduce or slow primary tumor growth or reduce or prevent metastasis). A therapeutically effective amount may also refer to the amount of the therapeutic agent that provides a therapeutic benefit in the treatment or management of a disease or disorder associated with a cell population. Further, a therapeutically effective amount with respect to a therapeutic agent of the invention means that amount of therapeutic agent alone, or in combination with other therapies, that provides a therapeutic benefit in the treatment, management, or amelioration of a disease or disorder associated with a targeted cell population.

As used herein, the terms “therapies” and “therapy” can refer to any protocol(s), method(s) and or agent(s) that can be used in the prevention, treatment, or management of diseases or disorders associated with a cell population. In certain embodiments, the terms “therapy” and “therapies” refer to cancer chemotherapy, radiation therapy, hormonal therapy, biological therapy, and/or other therapies useful for the treatment of cancer, infectious diseases, autoimmune and inflammatory diseases known to a physician skilled in the art.

As used herein, the terms “treat,” “treating” and “treatment” refer to the killing or suppression of cells that are related to a disease or disorder resulting from the administration of one or more prophylactic or therapeutic agents.

### 4. FIGURES

FIGS. 1A and 1B show the nucleotide sequence (SEQ ID NO:1) and amino acid sequence (SEQ ID NO:2), respectively, of mouse angiostatin.

FIG. 2 shows a schematic diagram of recombinant AAV (rAAV)-angiostatin construct in which CAG promoter, reporter gene, the 1.4-kb cDNA encoding mouse angiostatin (SEQ ID NO:1), wood chuck hepatitis B virus post-transcriptional regulatory element (WPRE), and poly A sequences, are inserted between the inverted terminal repeats (ITRs).

FIGS. 3A-3F show a long-term expression of angiostatin in hepatocytes after the transfusion of rAAV-angiostatin via portal vein. Overexpression of angiostatin in hepatocytes was detected by immunohistochemical analysis (A, B, C) and in situ hybridization (D, E, F). Representative liver sections were prepared 14 days following empty AAV treatment (A, D), 14 days (B, E) or 180 days (C, F) following AAV-angiostatin treatment and reacted with monoclonal antibody (mAb) against angiostatin (stained brown) or, hybridized with digoxigenin (DIG)-labeled antisense cRNA (100× magnification; stained green).

FIG. 4 shows the result of Western blotting in which the extracts from the homogenized liver cells of the mice transfused with rAAV-angiostatin were immunoblotted with anti-angiostatin antibody (Ab) or anti-beta-actin Ab (as an internal control). The mice were hepatectomized 2 days (Band 1), 14 days (Band 2), 28 days (Band 3), 60 days (Band 4), 90 days (Band 5) or 180 days (Band 6) following AAV-angiostatin transfusion.

FIGS. 5A-5C show the effects of gene transfer of rAAV-angiostatin via portal vein on liver metastatic tumors of both nodular and disseminated forms in terms of tumor volumes; relative areas of metastatic tumors; and % survival. (A) Liver nodular metastatic tumors were established by the injection of 2×10⁵ EL-4 tumors under the Glisson's capsule into the left lobe of the liver, followed by intraportal transfusion of 3×10¹¹ particles of rAAV-angiostatin virus. PBS and empty AAV virus served as controls. The mice were hepatectomized and volumes of tumors were measured 4 weeks after operation. Each point represents a single animal. The mean tumor volume is indicated by the large cross (P<0.01). (B) Disseminated liver metastatic tumors were established by intrasplenic injection of 2×10⁵ EL-4 tumor cells, followed by intraportal transfusion treatment. Six weeks after operation, all the mice were hepatectomized, the liver samples cryostated, and areas of tumors measured with a sigma Image Software. The mean relative area occupied by tumors is indicated by the large cross (P<0.01). (C) Disseminated liver metastatic cancer models were established by intrasplenic injection of 1×10⁶ EL-4 tumor cells, randomly followed by intraportal transfusion of PBS, empty AAV, or rAAV-angiostatin. The mice were observed twice weekly. The mice were sacrificed when they became moribund by pre-established criteria and their survival curves were plotted.

FIGS. 6A-6C show the inhibition of tumor vascularization, independently of Vascular Endothelial Growth Factor (VEGF), by rAAV-angiostatin treatment. EL-4 tumors were directly injected under the Glisson's capsule into the left lobe of the liver, followed by transfusion of PBS (A), empty AAV (B), or AAV-angiostatin(C), via portal vein. Four weeks after treatment, the mice were hepatectomized. The tumors were bisected, frozen, and stained with anti-CD31 antibody.

FIGS. 7A-7C show the effects of rAAV-angiostatin treatment on tumor vascularization (A and B) and the VEGF expression (C). Blood vessels stained with the anti-CD31 mAb were counted in blindly chosen random fields to record mean vessel density (A), or median distance to the nearest labeling for CD31 from an array point was recorded using the concentric circles methods (B). Significant difference (P<0.01; donated by stars) was observed between the tumors treated with rAAV-Angiostatin, and either PBS or empty AAV viruses. The transfusion of rAAV-angiostatin into the liver had no significant effect on the VEGF expression by the tumor cells as shown in Western Blotting using a VEGF-specific Ab (C) (Band1: PBS; Band 2: empty AAV; and Band 3: rAAV-angiostatin).

FIGS. 8A-8C show the apoptotic effect of rAAV-angiostatin using TUNEL. The rAAV-angiostatin treatment resulted in increase of apoptosis in tumor cells, but not in normal hepatocytes. EL-4 tumors were directly injected under the Glisson's capsule into the left lobe of the liver, followed by transfusion of rAAV-angiostatin virus particles (C), PBS (A), or empty AAV particles (B), via portal vein. Four weeks after treatment, the mice were hepatectomized. The liver tumors were bisected in horizontal plane and frozen. Slides were examined for apoptosis using TUNEL, and their adjacent sections were stained with haematoxylin/eosin in order to compare the apoptotic index (see below). The arrows point to the position of tumors in the liver.

FIG. 9 shows the comparison of apoptosis indices (AI) [(number of apoptotic cells/total number of nucleated cells)×100]. AI were significantly (noted with an asterisk) higher with rAAV-angiostatin than with PBS (P<0.001), or rAAV-angiostatin and empty AAV groups (P<0.01).

FIGS. 10A-10C show the transfection efficiency of AAV-B7.1. Parental EL-4 cells were incubated with AAV-B7.1 for 6 hours. FIG. 10A shows B7.1 protein expression on the surface of EL-4 cells (thick lines) and background staining with secondary antibodies (Abs) (light lines). FIG. 10B shows B7.1 protein expression on the surface of EL-4 cells transfected with the AAV-B7.1 vector following immunostaining with a specific anti-B7.1 monoclonal antibody (mAb) and FITC-labeled secondary Ab and subsequent visualization by fluorescence microscopy. EL-4 cells incubated with empty AAV vector were used as a control. FIG. 10C confirms B7.1 protein expression after AAV-B7.1 transfection as evidenced by Western blot analysis. The blot was stained with an anti-β-actin antibody to demonstrate equal loading of protein in each lane.

FIGS. 11A-E show that transfusion of AAV-angiostatin via a portal vein leads to long-term and persistent expression of angiostatin in hepatocytes. FIGS. 11A and 1C show liver sections prepared 14 days following treatment with empty AAV. FIGS. 11B and 11D show liver sections prepared 14 days following treatment with AAV-angiostatin. FIGS. 11A and 11C show low endogenous levels of angiostatin in hepatocytes treated with empty AAV detected by in situ hybridization and immunohistochemistry, respectively. FIGS. 11B and 11D show overexpression of angiostatin in hepatocytes treated with AAV-angiostatin detected by in situ hybridization and immunohistochemistry, respectively. A woodchuck hepatitis B virus post-transcriptional regulatory element (WPRE) RNA was stained blue with DIG-labeled antisense cRNA (indicated by arrows). Angiostatin protein was stained brown with an anti-angiostatin specific mAb. FIG. 11E confirms the expression of angiostatin in vivo by Western blot analysis with an anti-angiostatin mAb. Liver homogenates were prepared from hepatectomized mice 2 (lane 2), 14 (lane 3), 60 (lane 4), and 180 (lane 5) days following AAV-B7.1 transfusion. Liver homogenates prepared at day 60 from mice receiving empty AAV were used as a control (lane 1).

FIGS. 12A-12B show that AAV-B7.1 transfected EL-4 cells stimulate anti-tumor immunity. FIG. 12A shows the relative areas (%) occupied by tumors in the livers from mice challenged by intraportal injection of EL-4 cells transfected with either AAV-B7.1 or empty AAV. Mean relative area occupied by tumors is indicated by the large cross. FIG. 12B-shows the results from an in vitro CTL killing assay where splenocytes from mice vaccinated with AAV-B7.1 transfected EL-4 cells that were free of liver tumors were mixed with EL-4 cells transfected with either AAV-B7.1 or empty AAV at an effector to target (E:T) ratio of 100:, 50:1 and 10:1. Cytotoxicity assays were also performed in the presence of anti-B7.1 Ab. * indicates significant difference at P<0.01 from parental EL-4 cells transfected with empty AAV.

FIGS. 13A-13C show that the anti-tumor immunity generated by vaccination with AAV-B7.1 transfected EL-4 cells could be memorized. FIG. 13A shows that the anti-tumor CTL activity of splenocytes obtained from mice free of tumors 4 weeks after intraportal injection of AAV-B7.1 transfected EL-4 cells was augmented versus anti-tumor CTL activity of splenocytes from mice receiving empty AAV transfected EL-4 cells. The percentage cytotoxicity is plotted against various effector to target (E:T) ratios. FIG. 13B shows the relative areas (%) occupied by tumors in the livers from unvaccinated and vaccinated mice challenged by intraportal injection of EL-4 cells. FIG. 13C shows the relative areas (%) occupied by tumors in the livers from unvaccinated and vaccinated mice rechallenged by intraportal injection of parental EL-4 cells. Although vaccinated mice failed to resist the rechallenge, the growth of tumors metastasized to the liver was suppressed. * and ** indicate a significant and highly significant difference from control groups of mice at P<0.01 and P<0.001, respectively.

FIGS. 14A-14C show that synergism from vaccination with AAV-B7.1 transfected EL-4 cells and AAV-angiostatin therapy eradicates disseminated metastatic liver tumors and improves the survival of mice. FIG. 14A shows the relative areas (%) occupied by tumors in the livers from unvaccinated mice treated with empty AAV viruses (1) or AAV-angiostatin (3) and mice vaccinated with AAV-B7.1 transfected EL-4 cells and treated with empty AAV viruses (2) or mice vaccinated with AAV-B7.1 transfected EL-4 cells and treated with AAV-angiostatin (4). FIG. 14B shows the survival rate of unvaccinated mice treated with empty AAV viruses (1) or AAV-angiostatin (3) and mice vaccinated with AAV-B7.1 transfected EL-4 cells and treated with empty AAV viruses (2) or mice vaccinated with AAV-B7.1 transfected EL-4 cells and treated with AAV-angiostatin (4). Mice were observed thrice weekly, and were sacrificed when they became moribund by pre-established criteria. FIG. 14C shows representative photographs of livers with metastatic tumors from unvaccinated mice treated with empty AAV viruses (1) or AAV-angiostatin (3) and mice vaccinated with AAV-B7.1 transfected EL-4 cells and treated with empty AAV viruses (2) or mice vaccinated with AAV-B7.1 transfected EL-4 cells and treated with AAV-angiostatin (4). The arrows point to the tumors in the livers.

### 5. DETAILED DESCRIPTION OF THE INVENTION

5.1 Construction of Vector and Expression of Proteins

The present invention relates to nucleic acid molecules comprising sequences encoding angiostatin or B7.1 molecules. The present invention relates to nucleic acid molecules that encode and direct the expression of the angiostatin and B7.1 molecule in appropriate host cells.

Due to the inherent degeneracy of the genetic code, other polynucleotides comprising nucleotide sequences that encode the same amino acid sequence for angiostatin or B7.1 molecule may be used in the practice of the present invention. These include but are not limited to nucleotide sequences comprising all or portions of the coding region of the angiostatin or B7.1 gene which are altered by substitution of different codons that encode the same amino acid residue within the sequence, thus producing a silent change. Such nucleic acid molecule comprises a nucleic acid sequence which hybridizes to sequence or its complementary sequence encoding the angiostatin and/or B7.1 gene under stringent conditions. In one embodiment, the nucleic acid molecule that hybridizes to a complement of SEQ ID NO:1, 3 or 5 comprises at least 5, 10, 15, 20, 30, 40, 50, 60, 70, 80, 100, 120, 130, 150, 170, 180, 200, 300, 400, 500, 600, 700, 800, 900, 1,000, 1,100, 1,300, 1,500, 2,000, 2,500, or multiples thereof of nucleotides.

In certain embodiments, the nucleic acid molecule comprises a nucleic acid sequence that encodes both angiostatin and the costimulatory molecule B7.1. In one embodiment, the nucleic acid molecule comprises an AAV vector and a cytomegalovirus enhancer and beta-actin promoter (CAG promoter) which is operably linked to a nucleic acid sequence encoding angiostatin. In a specific embodiment, the nucleic acid molecule comprises an AAV vector and a CAG promoter which is operably linked to either the nucleotide sequence of SEQ ID NO:1 or a nucleotide sequence that encodes the amino acid sequence of SEQ ID NO:2.

The phrase “stringent conditions” as used herein refers to those hybridizing conditions that (1) employ low ionic strength and high temperature for washing, for example, 0.015 M NaCl/0.0015 M sodium citrate/0.1% SDS at 50° C.; or hybridization in 6× sodium chloride/sodium citrate (SSC), 0.5% SDS at about 68° C. followed by one or more washes in 2×SSC, 0.5% SDS at room temperature; or hybridization in 6×SSC at about 45° C. followed by one or more washes in 0.2×SSC, 0.1% SDS at about 50-65° C.; (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or (3) employ 50% formamide, 5×SSC (0.75 M NaCl, 0.075 M Sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS. The nucleic acid molecules comprising sequences encoding angiostatin or B7.1 molecules may be engineered, including but not limited to, alterations which modify processing and expression of the gene product. For example, to alter glycosylation patterns or phosphorylation, etc.

In certain embodiments, the nucleic acid molecules of the invention comprise a nucleotide sequence that encodes angiostatin and B7.1. In a specific embodiment, the nucleic acid molecule comprises a nucleotide sequence that comprises the nucleotide sequences of SEQ ID NOS:1 and 3. In a specific embodiment, the nucleic acid molecule comprises a nucleotide sequence that comprises the nucleotide sequences of SEQ ID NOS:1 and 5. In another specific embodiment, the nucleic acid molecule comprises a nucleotide sequence that encodes the amino acid sequences of SEQ ID NOS:2 and 4. In another specific embodiment, the nucleic acid molecule comprises a nucleotide sequence that encodes the amino acid sequences of SEQ ID NOS:2 and 6.

In order to express a biologically active angiostatin or B7.1 protein, the nucleotide sequence encoding angiostatin or B7.1 protein, respectively, is inserted into an appropriate expression vector, i.e., a vector which contains the necessary elements for the transcription and translation of the inserted nucleic acid molecule. The gene products as well as host cells or cell lines transfected or transformed with recombinant expression vectors are within the scope of the present invention.

Methods which are well known to those skilled in the art can be used to construct expression vectors containing the sequence that encodes the angiostatin or B7.1 molecule and appropriate transcriptional/translational control signals. These methods include in vitro recombinant DNA techniques, synthetic techniques and in vivo recombination/genetic recombination. See, for example, the techniques described in Sambrook et al., 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y.

A variety of host-expression vector systems may be utilized to express the angiostatin and/or B7.1 molecule. These include but are not limited to microorganisms such as bacteria transformed with recombinant bacteriophage DNA, plasmid DNA or cosmid DNA expression vectors; yeast transformed with recombinant yeast expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid); or animal cell systems.

The expression elements of each system vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter; EGR-1 promoter; and target specific promoter albumin) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter), from mammalian viruses (e.g., the adenovirus late promoter; the vaccinia virus 7.5K promoter), or avian cells (e.g., chicken beta-actin promoter) may be used; when generating cell lines that contain multiple copies of the chimeric DNA, SV40-, BPV- and EBV-based vectors may be used with an appropriate selectable marker.

In bacterial systems a number of expression vectors may be advantageously selected depending upon the use intended for the protein expressed. For example, when large quantities of protein are to be produced, vectors which direct the expression of high levels of protein products that are readily purified may be desirable. Such vectors include but are not limited to the pHL906 vector (Fishman et al. Biochem. 1994; 33: 6235-6243), the E. coli expression vector pUR278 (Ruther et al. EMBO J. 1983; 2: 1791), in which the protein coding sequence may be ligated into the vector in frame with the lacZ coding region so that a hybrid AS-lacZ protein is produced; pIN vectors (Inouye & Inouye. Nucleic Acids Res. 1985; 13: 3101-3109; Van Heeke & Schuster. J Biol. Chem. 1989; 264: 5503-5509); and the like.

Specific initiation signals may also be required for efficient translation of the nucleic acid molecule of the present invention. These signals include the ATG initiation codon and adjacent sequences. In cases where the entire gene, including its own initiation codon and adjacent sequences, is inserted into the appropriate expression vector, no additional translational control signals may be needed. However, in cases where the angiostatin or B7.1 protein coding sequence does not include its own initiation codon, exogenous translational control signals, including the ATG initiation codon, must be provided. Furthermore, the initiation codon must be in phase with the reading frame of the angiostatin or B7.1 protein coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al. Methods in Enzymol. 1987; 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. The presence of consensus N-glycosylation sites in the angiostatin or B7.1 protein may require proper modification for optimal function. Different host cells have characteristic and specific mechanisms for the post-translational processing and modification of proteins. Appropriate cell lines or host systems can be chosen to ensure the correct modification and processing of the protein. To this end, eukaryotic host cells which possess the cellular machinery for proper processing of the primary transcript, glycosylation, and phosphorylation of the angiostatin or B7.1 protein may be used. Such mammalian host cells include but are not limited to CHO, VERO, BHK, HeLa, COS, MDCK, 293, WI38, and the like.

For long-term, high-yield production of angiostatin and B7.1 proteins, stable expression is preferred. For example, cell lines which stably express the angiostatin or B7.1 protein may be engineered. Rather than using expression vectors which contain viral origins of replication, host cells can be transformed with a coding sequence controlled by appropriate expression control elements, such as promoter (e.g., chicken beta-actin promoter, EGR-1 promoter, and target specific promoter albumin), enhancer (e.g., CMV enhancer), transcription terminators, post-transcriptional regulatory element (e.g., WPRE), polyadenylation sites, etc., and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85:8047); and ODC (ornithine decarboxylase) which confers resistance to the ornithine decarboxylase inhibitor, 2-(difluoromethyl)-DL-omithine, DFMO (McConlogue L., 1987, In: Current Communications in Molecular Biology, Cold Spring Harbor Laboratory ed.).

The identity and functional activities of an angiostatin or B7.1 molecule can be readily determined by methods well known in the art. For example, antibodies to the protein may be used to identify the protein in Western blot analysis or immunohistochemical staining of tissues.

5.2 Pharmaceutical Compositions

The therapeutic agent of the invention can be incorporated into pharmaceutical compositions suitable for administration. Such compositions typically comprise the nucleic acid molecule; and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions comprising nucleic acid molecules of the invention. Such methods comprise formulating a pharmaceutically acceptable carrier with the therapeutic agent of the invention. Such compositions can further include additional active agents. Thus, the invention further includes methods for preparing a pharmaceutical composition by formulating a pharmaceutically acceptable carrier with the nucleic acid molecules of the invention and one or more additional active compounds.

A pharmaceutical composition of the invention is formulated to be compatible with its intended route of administration. Examples of routes of administration include parenteral, e.g., intra-arterial, intraportal, muscular, intravenous, intradermal, subcutaneous, transdermal (topical), transmucosal, intra-articular, intraperitoneal, and intrapleural, as well as oral, inhalation, and rectal administration. In a preferred embodiment, the route of administration is intraportal, e.g., via a portal vein. In another preferred embodiment, the route of administration is muscular, e.g., at the deltoid site, dorsogluteal site, vastus lateralis site, and ventrogluteal site. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy injectability with a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a nucleic acid molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient, such as starch or lactose; a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in cell cultures or animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography. For the use of animal models to determine optimal dosage, see, for example, Section 6.2, infra.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a therapeutic agent, such as nucleic acid molecules, can include a single treatment or, preferably, can include a series of treatments. It will also be appreciated that the effective dosage of nucleic acid molecule used for treatment may increase or decrease over the course of a particular treatment. Changes in dosage may result and become apparent from the results of diagnostic assays as described herein. The exact formulation, route of administration and dosage can be chosen by the individual physician in view of the patient's condition. (See, e.g., Fingl et al., 1975, In: The Pharmacological Basis of Therapeutics, Ch.1, p.1).

The nucleic acid molecules of the invention can be inserted into vectors and used as gene therapy vectors. Methods of delivering gene therapy vectors to a subject include: intravenous injection, local administration (U.S. Pat. No. 5,328,470) or by stereotactic injection (see, e.g., Chen, et al., 1994, Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. With regard to gene therapy, see further discussion in section 5.3.4.

5.3 Therapeutic/Prophylactic Methods Using Nucleic Acid Molecules of the Invention

The present invention is directed to therapeutic or prophylactic method which leads to the treatment or prevention of a disease or disorder that is associated with aberrant activity of a particular cell population. The disease or disorder is treatable or preventable by reducing the number of cells or to delay or minimize the proliferation of cells. The present invention also provides methods of preventing recurrence of tumor or cancer.

5.3.1 Cancer

Cancers and related disorders that can be treated or prevented by methods and compositions of the present invention include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America)

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal orignin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosacoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, liver, lung, skin, pancreas, or uterus. In other specific embodiments, sarcoma, melanoma, or leukemia is treated or prevented.

5.3.2 Therapeutic/Prophylactic Administration

The invention provides methods of preventing and treating cancer, tumor, or the recurrence of cancer or tumor by administrating to an animal (e.g., cows, pigs, horses, chickens, cats, dogs, humans, etc.) an effective amount of the polynucleotides of the invention. The polynucleotides of the invention may be administered to a subjectper se or in the form of a pharmaceutical composition for the treatment and prevention of cancer. In a specific embodiment, the polynucleotides of the invention are administered by intraportal injection. In another specific embodiment, the polynucleotides of the invention are administered by muscular injection.

In certain embodiments, therapeutic or prophylactic composition of the invention is administered to a mammal, preferably a human, concurrently with one or more other therapeutic or prophylactic composition useful for the treatment of diseases or disorders. In one embodiment, the AAV-B7.1 vector is administered concurrently with the AAV-angiostatin vector. The term “concurrently” is not limited to the administration of prophylactic or therapeutic composition at exactly the same time, but rather it is meant that the composition of the present invention and the other agent are administered to a mammal in a sequence and within a time interval such that the composition comprising the polynucleotides can act together with the other composition to provide an increased benefit than if they were administered otherwise. For example, each prophylactic or therapeutic composition (e.g., chemotherapy, radiation therapy, hormonal therapy, biological therapy, embolization, or chemoembolization therapies) may be administered at the same time or sequentially in any order at different points in time; however, if not administered at the same time, they should be administered sufficiently close in time so as to provide the desired therapeutic or prophylactic effect. Each therapeutic composition can be administered separately, in any appropriate form and by any suitable route. In other embodiments, the composition of the present invention is administered before, concurrently or after surgery. Preferably the surgery completely removes localized tumors or reduces the size of large tumors. Surgery can also be done to relieve pain. In various embodiments, the prophylactic or therapeutic compositions are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In other embodiments, the prophylactic or therapeutic compositions are administered at about 30 minutes, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 1 to 2 days apart, at about 2 to 4 days apart, at about 4 to 6 days apart, at about 1 week part, at about 1 to 2 weeks apart, or more than 2 weeks apart. In preferred embodiments, the prophylactic or therapeutic compositions are administered in a time frame where both compositions are still active. In a specific embodiment, a first AAV-B7.1 vector, the AAV-angiostatin vector, or the AAV-B7.1/angiostatin vector is administered 4 weeks before a second AAV-B7.1 vector, AAV-angiostatin vector, and/or AAV-B7.1/angiostatin vector is administered. One skilled in the art would be able to determine such a time frame by determining the half life of the administered compositions.

In a specific embodiment, the AAV-B7.1 and AAV-angiostatin vectors are both administered by intraportal injection. In another specific embodiment, the AAV-B7.1 and AAV-angiostatin vectors are both administered by muscular injection. In another specific embodiment, the AAV-B7.1 vector is administered by intraportal injection and the AAV-angiostatin vector is administered by muscular injection. In yet another specific embodiment, the AAV-B7.1 vector is administered by muscular injection and the AAV-angiostatin vector is administered by intraportal injection.

In certain embodiments, the prophylactic or therapeutic compositions of the invention are cyclically administered to a subject. Cycling therapy involves the administration of a first composition for a period of time, followed by the administration of a second composition and/or third composition for a period of time and repeating this sequential administration. Cycling therapy can reduce the development of resistance to one or more of the therapies, avoid or reduce the side effects of one of the therapies, and/or improves the efficacy of the treatment.

In certain embodiments, prophylactic or therapeutic compositions are administered in a cycle of less than about 3 weeks, about once every two weeks, about once every 10 days or about once every week. One cycle can comprise the administration of a therapeutic or prophylactic composition by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In yet other embodiments, the therapeutic and prophylactic compositions of the invention are administered in metronomic dosing regiments, either by continuous infusion or frequent administration without extended rest periods. Such metronomic administration can involve dosing at constant intervals without rest periods. The dosing regimens encompass the chronic daily administration of relatively low doses for extended periods of time. In preferred embodiments, the use of lower doses can minimize toxic side effects and eliminate rest periods. In certain embodiments, the therapeutic and prophylactic compositions are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimalized by the skilled physician.

The dosage amounts and frequencies of administration provided herein are encompassed by the terms therapeutically effective and prophylactically effective. The dosage and frequency further will typically vary according to factors specific for each patient depending on the specific therapeutic or prophylactic composition administered, the severity and type of disease or disorder, the route of administration, as well as age, body weight, response, and the past medical history of the patient. Suitable regimens can be selected by one skilled in the art by considering such factors and by following, for example, dosages reported in the literature and recommended in the Physician 's Desk Reference (56^{th} ed., 2002).

Various delivery systems are known and can be used to administer the therapeutic or prophylactic composition of the present invention, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic composition of the invention include, but are not limited to, parenteral administration (e.g., intradermal, intramuscular, intraperitoneal, intravenous and subcutaneous), epidural, and mucosal (e.g., intranasal and oral routes). In a specific embodiment, prophylactic or therapeutic composition of the invention are administered intramuscularly, intravenously, or subcutaneously. The prophylactic or therapeutic composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic composition of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapeutic or prophylactic composition of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one skilled in the art can be used to produce sustained release formulations comprising one or more therapeutic composition of the invention. See, e.g., U.S. Pat. No. 4,526,938, PCT publication WO 91/05548, PCT publication WO 96/20698,. Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

5.3.3 Other Therapeutic/Prophylactic Agents

According to the invention, therapy by administration of the polynucleotides may be combined with the administration of one or more therapies such as, but not limited to, chemotherapies, radiation therapies, hormonal therapies, biological therapies/immunotherapies, embolization, and/or chemoembolization therapies.

In a specific embodiment, the methods of the invention encompass the administration of one or more angiogenesis inhibitors such as but not limited to: antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Additional examples of anti-cancer agents that can be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytotoxic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. These two agents are particularly useful when used in methods employing thalidomide and a topoisomerase inhibitor.

Other anti-cancer agents that are useful for the methods of the present invention include herbs, herbal extracts or Chinese medicine that treat, manage and prevent neoplastic diseases. These remedies may be used in combination with the vector of the present invention for the treatment of cancer.

5.3.4 Gene Therapy

The present invention provides methods for the treatment or prevention of cancer, and tumor comprising administering nucleic acid molecules of the present invention encoding angiostatin or B7.1. In a specific embodiment, nucleic acid molecules comprising sequences encoding angiostatin or B7.1 are administered to treat or prevent cancer, by way of gene therapy. Gene therapy refers to therapy performed by the administration to a subject of an expressed or expressible nucleic acid. In this embodiment of the invention, the nucleic acid molecules produce their encoded protein that mediates a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11 (5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In one aspect, a composition comprising nucleic acid molecules comprising nucleic acid sequences encoding angiostatin or B7.1 in expression vectors of the present invention are administered to suitable hosts. The expression of nucleic acid sequences encoding angiostatin or B7.1 may be regulated by any inducible, constitutive, or tissue-specific promoter known to those of skill in the art. In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

In a particular embodiment, nucleic acid molecules encoding angiostatin or B7.1 are flanked by regions that promote homologous recombination at a desired site in the genome, thus providing for intrachromosomal expression of said coding regions (Koller and Smithies, 1989, Proc. Natl. Acad. Sci. USA 86:8932-8935; Zijlstra et al., 1989, Nature 342:435-438).

Delivery of the nucleic acids into a patient may be either direct, in which case the patient is directly exposed to the nucleic acid molecules or nucleic acid molecule-carrying vectors, or indirect, in which case, cells are first transformed with the nucleic acid molecules in vitro, then transplanted into the patient. These two approaches are known, respectively, as in vivo or ex vivo gene therapy.

In a specific embodiment, the nucleic acid molecules are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid molecules to avoid lysosomal degradation. In yet another embodiment, the nucleic acid molecules can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid molecules can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, viral vectors are used to express nucleic acid sequences. For example, a retroviral vector can be used (see Miller et al., 1993, Meth. Enzymol. 217:581-599). These retroviral vectors have deleted retroviral sequences that are not necessary for packaging of the viral genome and integration into host cell DNA. The nucleic acid molecules encoding the nucleic acid sequences to be used in gene therapy are cloned into one or more vectors, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783. In a preferred embodiment, adenovirus vectors are used. Adeno-associated virus (AAV) has also been proposed for use in gene therapy (Walsh et al., 1993, Proc. Soc. Exp. Biol. Med. 204:289-300; U.S. Pat. No. 5,436,146).

Most preferable viral vectors for the present invention are adeno-associated viral (AAV) vectors. AAV vector leads to persistent (>6 months) expression of a transgene in both gut epithelial cells and hepatocytes, resulting in long-term phenotypic recovery in a diabetic animal model (Xu, RA et al., 2001, Perarolly transduction of diffuse cells and hepatocyte insulin leading to euglycemia in diabetic rats, Mol Ther 3:S180; During, MJ et al., 1998, Perarolly gene therapy of lactose intolerance using an adeno-associated virus vector, Nature Med. 4:1131-1135; During MJ et al., 2000, An oral vaccine against NMDAR1 with efficacy in experimental stroke and epilepsy, Science 287:1453-1460).

AAV is a nonpathogenic, helper-dependent member of the parvovirus family with several major advantages, such as stable integration, low immunogenicity, long-term expression, and the ability to infect both dividing and non-dividing cells. It is capable of directing long-term transgene expression in largely terminally differentiated tissues in vivo without causing toxicity to the host and without eliciting a cellular immune response to the transduced cells (Ponnazhagan S et al., 2001, Adeno-associated Virus for Cancer Gene Therapy, Cancer Res 61:6313-6321; Lai CC et al., 2001, Suppression of choroidal neovascularization by adeno-associated virus vector expressing angiostatin, Invest Ophthalmol Vis Sci 42(10):2401-7; Nguyen JT et al., 1998, Adeno-associated virus-mediated delivery of antiangiogenic factors as an antitumor strategy, Cancer Research 58:5673-7).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In one embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid molecules to the cell, so that the nucleic acid molecules comprising nucleic acid sequences are expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences of the present invention encoding angiostatin or B7.1 are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

5.4 Demonstration of Therapeutic/Prophylactic Utility

The compositions of the invention are preferably tested in vitro, and then in vivo for the desired therapeutic or prophylactic activity, prior to use in humans. For example, in vitro assays to demonstrate the therapeutic or prophylactic utility of a composition include, the effect of a composition on a cell line, particularly one characteristic of a specific type of cancer, or a patient tissue sample. The effect of the composition on the cell line and/or tissue sample can be determined utilizing techniques known to those of skill in the art including, but not limited to, rosette formation assays and cell lysis assays. Specifically, liver cancer cell line, breast cancer cell line, such as MDA-MB-231, lymphoma cell line, such as U937, and colon cancer cell line, such as RKO may be used to assess the therapeutic effects of the polynucleotides encoding angiostatin or B7.1 protein. Techniques known to those skilled in the art can be used for measuring cell activities. For example, cellular proliferation can be assayed by ³H-thymidine incorporation assays and trypan blue cell counts.

As a specific example for testing a therapeutic or prophylactic activity of the therapeutic agent of the present invention, chicken chorioallantoic membrane (CAM) assay can be used. This is a secondary and independent assay of angiostatin activity. The one-day-old fertilized eggs were incubated for three days in the water-jacketed incubator (38° C., 85% humidity). The eggs were cracked and the chick embryos with intact yolks were placed in plastic Petri dishes containing 10 ml of RPMI-1640 medium (38° C., 85% humidity, 3% of CO₂). After 3 days of incubation, the methylcellulose disk containing inhibitor was implanted on the CAMs of the individual embryos. After 48h of incubation, CAM of individual embryo was analyzed for formation of avascular zones and photographed. The angiostatic effect of angiostatin was determined as a percentage of the area of blood vessels under the methylcellulose disks (3-5 eggs for each concentration) in relation to the non-treated areas.

In another specific example, the inhibition of tumor vascularity by the therapeutic agent of the present invention can be assessed by counting the number of blood vessels, of a tissue sample from a subject treated with the therapeutic agent, which are stained with a specific antibody against endothelial cells (e.g., anti-CD31 antibody) and compare with that of controls.

In yet another specific example, the expression of the therapeutic agent of the present invention can be detected by in situ hybridization using a specific probe, or by Western blotting or immunohistochemical staining using specific antibodies.

In yet another specific example, the therapeutic or prophylactic activity of the present therapeutic agent can be assessed by counting the number of apoptotic cells in the treated tissue sample using TUNEL staining method (Hensey C et al., 1998, Program cell death during Xenopus development: a spatio-temporal analysis, Dev Biol 203:36-48; Veenstra, G J et al., 1998, Non-cell autonomous induction of apoptosis and loss of posterior structures by activation domain-specific interactions of Oct-1 in the Xenopus embryo, Cell Death Differ 5:774-84) and compare with that of control samples.

Test composition can be tested for their ability to reduce tumor formation in patients (i.e., animals) suffering from cancer. Test compositions can also be tested for their ability to alleviate of one or more symptoms associated with cancer. Further, test compositions can be tested for their ability to increase the survival period of patients suffering from cancer. Techniques known to those of skill in the art can be used to analyze test to function of the test compositions in patients.

In various embodiments, with the invention, in vitro assays which can be used to determine whether administration of a specific composition is indicated, include in vitro cell culture assays in which a patient tissue sample is grown in culture, and exposed to or otherwise administered a composition, and the effect of such composition upon the tissue sample is observed. Specifically, cytotoxic effects of the expressed proteins may be assessed by Promega's Cell Titer 96 Aqueous Cell Proliferation assay and Molecular Probe's Live/Dead Cytotoxicity Kit.

Compositions for use in therapy can be tested in suitable animal model systems prior to testing in humans, including but not limited to rats, mice, chicken, cows, monkeys, rabbits, etc. For in vivo testing, prior to administration to humans, any animal model system known in the art may be used.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention also provides kits that can be used in the above methods. In one embodiment, a kit comprises the nucleic acid molecules in one or more containers.

In certain embodiments, the kits of the invention contain instructions for the use of the nucleic acid molecules for the treatment, prevention of cancer, viral infections, or microbial infections.

The invention is further defined by reference to the following example describing in detail the clinical trials conducted to study the efficacy and safety of the arsenic trioxide compositions of the invention.

The following examples illustrate the preparation and use of the AAV-angiostatin vector A and AV-B7.1 vector of the present invention. These examples should not be construed as limiting.

### 6. EXAMPLE 1

6.1 Generation of rAAV-Angiostatin

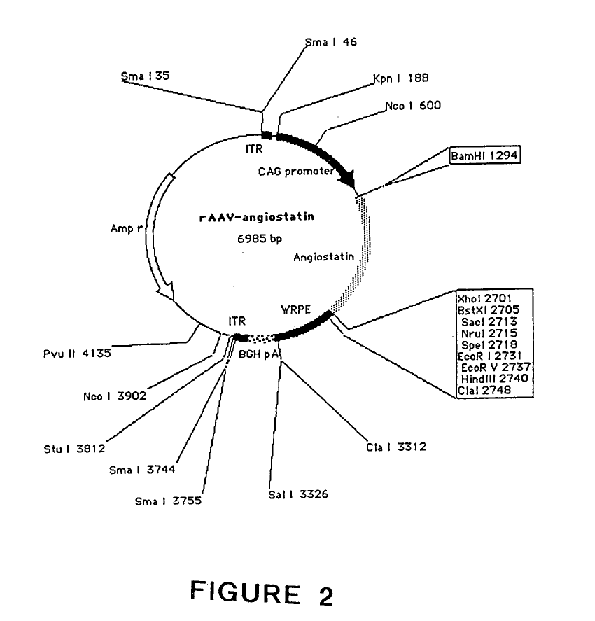

In the expression plasmid vector, chicken beta-actin promoter with cytomegalovirus (CMV) enhancer (CAG promoter) (Xu L. et al. CMV-beta-actin promoter directs higher expression from an adeno-associated viral vector in the liver than the cytomegalovirus or elongation factor 1 alpha promoter and results in therapeutic levels of human factor X in mice. Hum Gene Ther. 2001; 12(5): 563-7), reporter gene, the 1.4-kb cDNA encoding full length of mouse angiostatin (SEQ ID NO:1) consisting of the signal peptide and first four kringle regions of mouse plasminogen, and poly A sequences, were inserted between the inverted terminal repeats (ITRs) using appropriate restriction enzymes (see FIG. 2). A woodchuck hepatitis B virus post-transcriptional regulatory element (WPRE) was inserted into this construct to boost expression levels (Donello J. et al., Woodchuck hepatitis virus contains a tripartite post-transcriptional regulatory element. J. Virol. 1998; 72: 5085-5092; Xu R. A. et al. Quantitative comparison of expression with adeno-associated virus (AAV-2) brain-specific gene cassettes. Gene Ther. 2001; 8: 1323-1332). Plasmids were prepared using Qiagen plasmid purification kits.

AAV particles were generated by a three-plasmid, helper-virus free packaging method (Donello J. et al. 1998, supra; Xiao W. et al. Route of administration determines induction of T cell independent humoral response to adeno-associated virus vectors. Mol Ther. 2000; 1(4): 323-9) with some modification. The 293 cells were transfected with rAAV-angiostatin, and the helper pFd, H22 using the calcium phosphate precipitation method. The cells were harvested 70 hours after transfection and lysed by incubation with 0.5% deoxycholate for 30 min at 37° C. in the presence of 50 units/ml Benzonase (Sigma, St. Louis, Mo.). After centrifugation at 5000 g, the cells were filtered with a 0.45-μm Acrodisc syringe filter to remove any particulate cellular matter for a heparin column. The rAAV particles were isolated by affinity chromatography with a little modification. The peak virus fraction was dialyzed against 100 mM NaCl, 1 mM MgCl₂ and 20 mM sodium mono- and di-basic phosphate buffer at pH 7.4. A portion of the samples was subjected to quantitative PCR analysis using the AB Applied Biosystem, to quantify genomic titer. The PCR TaqMan® assay was a modified dot-blot protocol, whereby AAV was serially diluted and sequentially digested with DNAse I and Proteinase K. Viral DNA was extracted twice with phenol-chloroform to remove proteins, and then precipitated with 2.5 equivalent volumes of ethanol. A standard amplification curve was set up at a range from 10² to 10⁷ copies and the amplification curve corresponding to each initial-template copy number was obtained. Viral particles were reconfirmed by a commercially available analysis kit (Progen, Germany). The viral vector was stored at −80° C. prior to animal experiments.

6.2 AAV-Mediated Antiangiogenic Gene Therapy in Mice

6.2.1 Mice, Cell Lines and Antibodies

Male C57BL/6 mice (H-2b), 6-8 weeks old, were obtained from the Laboratory Animal Unit of University of Hong Kong. The syngeneic (H-2b) EL-4 thymic lymphoma cell line was purchased from the American Type Culture Collection (Rockville, Md., USA). The cells were cultured at 37° C. in DMEM medium (Gibco BRL, Grand Island, N.Y.) supplemented with 10% fetal calf serum, 50 U/ml penicillin/streptomycin, 2 mM L-glutamine, and 1 mM pyruvate. The anti-plasminogen mAb, rabbit polyclonal anti-VEGF antibody, and anti-CD31 antibody MEC13.3, were purchased from Calbiochem-Novabiochem Corporation, Lab Vision Corporation, and Pharmingen (CA, USA), respectively.

6.2.2 Experimental Protocol

All surgical procedures and care administered to the animals were in accordance with the institutional guidelines. Animals were randomly assigned to treatment. Each group contained 10 mice. The nodular and disseminated tumor models consistently yielded tumors in at least 90-95% animals. An equal volume of PBS and equal particle number of empty AAV virus or AAV viral vector containing reporter gene served as controls.

6.2.2.1 Induction of Liver Nodular Tumors

After anesthetization of the mice, the liver was surgically exposed and 2×10⁵ EL-4 tumor cells were injected under the Glisson's capsule into the left lobe of the liver with a 30-G needle or via the portal vein. One week later, 3×10¹¹ particles of rAAV-angiostatin virus were injected via portal vein. Hemostatasis was performed and the abdominal cavity was closed. Five weeks after the operation, the mice were killed, and the tumors in the left lobe of the liver were excised and measured with calipers in the two perpendicular diameters (a and b, respectively). The tumor volume was calculated according to the formula (a×b×27π)/6, as previously described (Auerbach R. et al. Regional differences in the incidence and growth of mouse tumors following intradermal or subcutaneous inoculation. Cancer Res. 1978; 38: 1739-1744).

6.2.2.2 Induction of Disseminated Live Metastatic Tumors

After anesthetization of the mice, the spleen was surgically exposed and completely exteriorize after separation of the short gastric vessels and gastrosplenic ligament. Firstly, 2×10⁵ EL4 tumor cells were slowly injected into the spleen with a 30-G needle. After a delay of approximately 5 minutes to allow the tumor cells to enter the portal circulation, splenectomy was performed after ligature of splenic pedicle. Secondly, 3×10¹¹ particles of rAAV-angiostatin virus were injected via portal vein. Hemostatasis was performed and the abdominal cavity was closed. Six weeks after the operation, the mice were killed and the livers excised. The livers were then frozen and cryostated to prepare transverse 10-μm sections made at 5 different levels to cover the entire liver. The sections were mounted and stained with hematoxylin and eosin. The entire liver and tumor areas were measured and examined under a microscope using a sigma software program. The relative areas occupied by the tumors were calculated in accordance with the formula: (total tumor areas/liver area)×100.

6.2.2.3 Survival Studies

Tumor models were generated as disseminated liver metastasis by intrasplenic injection of 1×10⁶ EL-4 tumor cells, followed by intraportal injection of 3×10¹¹ particles of AAV-Angiostatin. The animals were weighed three times weekly and assessed. Moribund mice were euthanized according to pre-established criteria; namely the presence of two or more of the following premoid conditions: gross ascites, palpable tumor burden greater than 2 cm, dehydration, lethargy, emaciation, and weight loss greater than 20% of the initial body weight.

6.3. Immunohistolgic Analysis

Cryosections (10 μm) prepared from the liver or tumors following intraportal AAV transfusion, underwent overnight incubation with specific Abs. The sections were subsequently incubated for 30 min with appropriate secondary antibodies (VECTASTAIN® Universal Quick kit, Vector Laboratories, Burlingame, Calif.), and developed with Sigma FAST DAB (3,3′-diaminobenzidine tetrahydrochloride) and CoCl₂ enhancer tablets (Sigma, St. Louis, Mo.). The sections were then counterstained with Mayer's hematoxylin.

6.4. In Situ Hybridization

Liver sections were fixed for 7 min in 4% formaldehyde and washed in PBS for 3 min and in 2×SSC for 10 min. The dehydrated sections were hybridized at 60° C. overnight with a probe solution according to in situ hybridization protocol (Ambion, Austin). The slides were washed with 4×SSC and incubated in RNAse digestion solution at 37° C. for 30 min. Slides were then washed with decreasing concentrations of SSC at room temperature at 5-min intervals with gentle agitation. The slides were then dehydrated with increasing concentrations of ethanol. Hybridization was detected by the kit, VECTASTAIN® ABC (Vector Laboratories, Burlingame, Calif.) and BCIP/NBT.

6.5 Western Blotting

Samples after the treatment were excised, minced and homogenized in a protein lysate buffer. Tissue or cell debris was removed by centrifugation at 10,000 g for 10 min at 4° C. Tumor lysates from each group of mice were pooled and the protein content determined. Protein samples (100 mg) were resolved on 10% polyacrylamide SDS gels and electrophoretically transferred to nitrocellulose Hybond™ C extra membranes (Amersham Life Science, England). After the membranes were blocked with 5% BSA, blots were incubated with specific primary Abs, followed by horseradish peroxidase-conjugated secondary antibodies, developed by enhanced chemiluminescence (Amersham International plc, England), and exposed to an X-Ray film. Band densities were quantified using Sigma ScanPro software.

6.6 Assessment of Vascularity

The methodology for determining tumor vascularity has been described previously (Sun X. et al. Angiostatin enhances B7.1-mediated cancer immunotherapy independently of effects on vascular endothelial growth factor expression. Cancer Gene Therapy 2001; 8: 719-727). Briefly, 10-mm frozen tumor sections prepared from liver nodular tumors 4 weeks after the treatment were immunostained with the anti-CD31 antibody, as described above. Stained blood vessels were counted in blindly chosen five random fields (0.155 mm²) at 40× magnification, and the mean of the highest three counts was calculated. The concentric circles method (Heather E. R. et al. HIF-1 a is required for solid tumor formation and embryonic vascularization. EMBO J. 1998; 17: 3005-3015; Kayar S. R. et al. Evaluation of the concentric-circles method for estimating capillary-tissue diffusion distances, Microvascular Res. 1982; 24: 342-353) was also used to assess vascularity.

6.7 In Situ Detection of Apoptotic Cells

Serial sections of 6-mm thickness were prepared from tumors 4 weeks following the treatment. TUNEL staining of sections was performed using an in situ cell death detection kit from Roche Molecular Biochemicals, Germany. Briefly, frozen sections were fixed with 4% paraformaldehyde solution, permeabilized with a solution of 0.1% Triton-X100 and 0.1% sodium citrate, incubated with TUNEL reagent for 60 min at 37° C., and examined by fluorescence microscopy. Adjacent sections were counterstained with hematoxylin and eosin. The total numbers of apoptotic cells in 10 randomly selected fields were counted. The apoptotic index was calculated as the percentage of positively stained cells (i.e., apoptotic cells); namely AI=(number of apoptotic cells/total number of nucleated cells)×100.

6.8 Statistical Analysis

For the tumor volumes and relative areas occupied by tumors, Kruskal-Wallis tests were performed to test the effect of treatment. For survival data, log rank tests were performed to test the effect of treatment. For other data, results were expressed as mean values±standard deviation (s.d.), and a Student's t test was used for evaluating statistical significance. P values were considered to be statistically significant when less than 0.05.

6.9 Results

6.9.1 Long-Term and Persistent Expression of Angiostatin in Liver After rAAV-Angiostatin Portal Vein Transfusion

One of the main advantages of rAAV is its ability to mediate long-term transgene expression. Injection of a recombinant rAAV-angiostatin vector via a portal vein successfully hemostatasis to a long-term expression of the exogenous gene in the liver for up to 6 months.

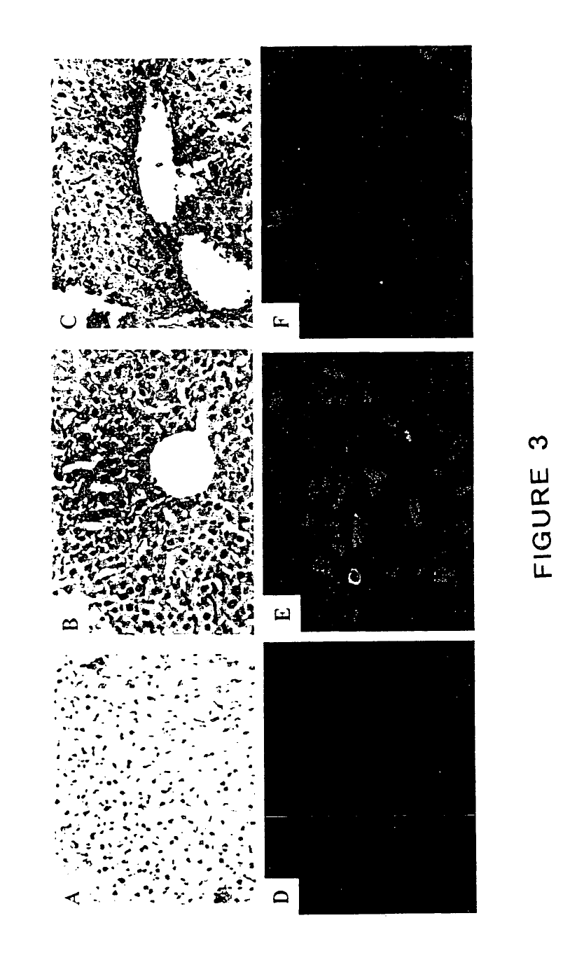

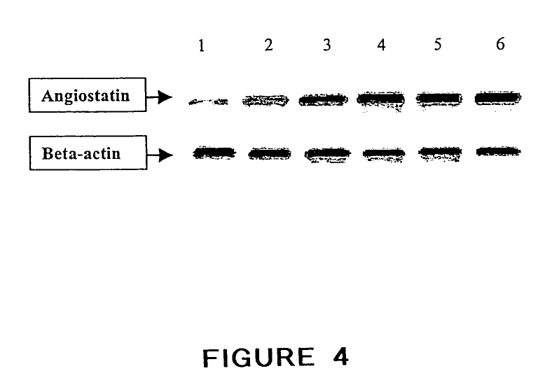

To analyze the efficiency of the gene-transfer, the liver samples were collected at 2, 14, 28, 60, 90 and 180 days after intraportal injection of rAAV-angiostatin. The expression of angiostatin in the liver was confirmed by immunohistochemistry, in situ hybridization and western blotting. As shown in FIG. 3, in situ over-expression of angiostatin was clearly detectable 14 days following gene transfer (FIG. 3B) and it persisted for 180 days following gene transfer (FIG. 3C), compared to only 2 days in the case of controls which were treated with empty AAV (A). As angiostatin is a fragment of plasminogen, which is an endogenous protein and detectable by anti-angiostatin Ab, the results were further confirmed by in situ hybridization with the DIG RNA labeling kit (FIGS. 3D, 3E, and 3F, which correspond to the liver sections of FIGS. 3A, 3B and 3C, respectively). The present inventors have previously reported that peroral transduction of AAV-insulin vector led to a gradual increase in transgenic insulin in hepatocytes over 3 months, after which a plateau was reached (Xu, RA, et al., 2001, supra; During et al., 2000, supra). In the case of intraportal transfusion of AAV-angiostatin, the expression of transgenic angiostatin in hepatocytes rose to high level in one month, increased to peak level in two months, and then was stabilized for six months. The samples were from mice hepatectomized at 2 days (Band1), 14 days (Band 2), 28 days (Band 3), 60 days (Band 4), 90 days (Band 5) or 180 days (Band 6) following AAV-angiostatin transfusion (see FIG. 4).

6.9.2 Suppression of Liver Metastatic Nodular Tumors and Disseminated Tumors

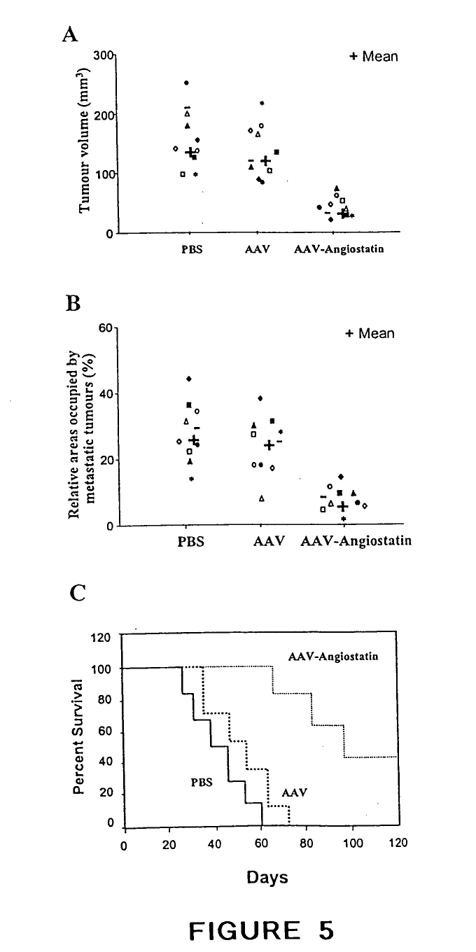

To analyze the therapeutic potential of the intraportal-vein injection of rAAV-angiostatin in respect of nodular liver tumors, EL-4 tumor cells were injected into the left lobe of the livers in 30 mice, each of which, then, randomly received an intraportal-vein injection of PBS (n=10), empty AAV (n=10), or rAAV-angiostatin viruses (n=10). Four weeks later, all the mice underwent hepatectomy. The volumes of liver tumors in each group are presented in FIG. 5A. The mean volume of left lobe tumors was 149.2 mm² and 127.5 mm² in the treatment groups which received PBS and empty AAV, respectively. The slight difference between these two groups was not statistically significant (P>0.05). In contrast, the mean volume of the left lobe tumors in the group treated with rAAV-angiostatin was only 40.3 mm², which was a 72% and 68% decrease in the tumor volumes of the groups treated with PBS and empty AAV, respectively. The results differed significantly from the cases treated with either PBS (P<0.001) or empty AAV (P<0.01).

To analyze the therapeutic potential of intraportal vein injection of rAAV-angiostatin in respect of disseminated hepatic metastatic tumors, EL-4 tumor cells were injected into the spleen of mice (n=30), and splenectomy was carried out. The mice then randomly underwent intraportal vein injection of PBS (n=10), empty AAV (n=10), or rAAV-angiostatin viruses (n=10). Six weeks later, the mice were killed and hepatectomized. The livers were cryostated transversely. The areas occupied by the tumors in the livers are illustrated in FIG. 5B. The mean relative areas occupied by tumors in the livers were 26.5%, 24.0% and 7.3% in PBS, empty AAV, and rAAV-angiostatin groups, respectively. There was no significant difference between the PBS- and empty AAV-treated groups (P>0.05). However, the rAAV-angiostatin treatment resulted in 72% and 71% reduction of the relative area occupied by tumors compared to PBS- and empty AAV-treated groups, respectively, demonstrating the statistically significant difference between rAAV-angiostatin-treated group and either of the control groups (each P<0.001).

6.9.3 rAAV-Angiostatin Improved Survival Rate of Mice With Liver Metastasises

The survival rate of the mice with liver metastasis which were treated with rAAV-angiostatin was further studied to investigate whether this treatment could result in a survival benefit for mice. Although the intrahepatic model enables accurate measurements of tumor sizes, the intrasplenic model, which more closely resembles the clinical situation, results in multiple liver metastasises via the portal system and can be better assessed by the survival rate. Thirty C57BL mice were intrasplenically injected with 1×10⁶ EL-4 tumor cells, then received intraportal injection of 3×10¹¹ particles of rAAV-angiostatin (n=10), PBS (n=10), or empty AAV (n=10), the latter two serving as controls. Treatment with AAV-angiostatin resulted in a profound and statistically significant improvement in the survival of mice intrasplenically challenged with tumor cells. Four of the ten mice in this group survived more than 80 days after tumor cell inoculation, whereas all the control mice in both the PBS and the empty AAV-treated groups died. Median survival time for the mice treated with PBS was 25 days and that for the mice treated with empty AAV was 29 days. There was no significant difference between these two groups (P>0.1). However, the median survival time for the mice treated with AAV-angiostatin was 58 days, which was a statistically significant difference from those of the PBS-treated group and empty AAV-treated group (each P<0.01) (see FIG. 5C), respectively.

6.9.4 Inhibition of Tumor Vascularization Independent of Endothelial Vascular Growth Factor

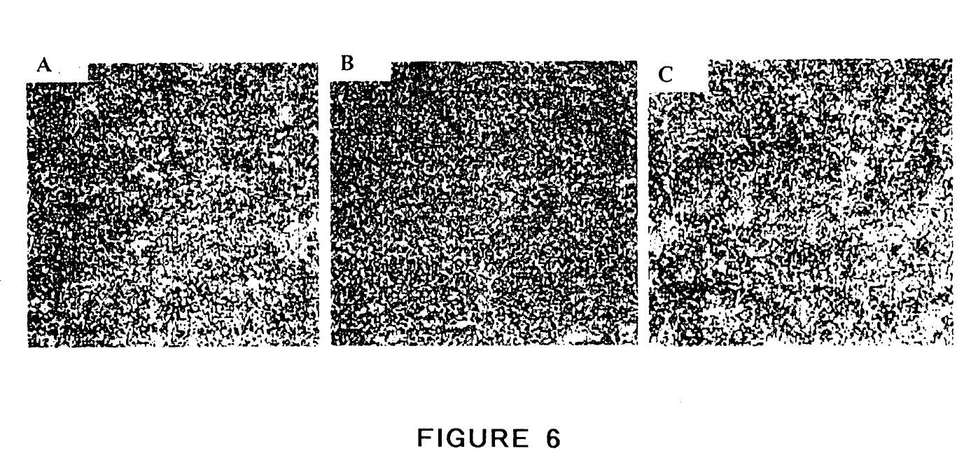

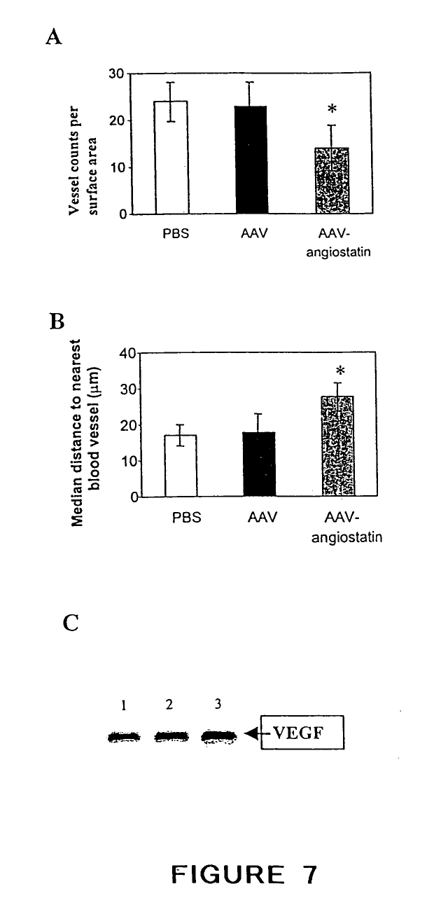

The transfusion of AAV-angiostatin via the portal vein resulted in inhibition of vascularization of liver nodular metastatic tumors. The nodular tumors established in the left lobe of the livers were removed 4 weeks following rAAV-angiostatin injection, cryostated into 10 μm sections, and stained with an anti-CD31 antibody. Representative pictures from mice treated with PBS (A), empty AAV (B), and AAV-angiostatin (C) are shown in FIG. 6. The rAAV-angiostatin therapy resulted in a significantly reduced tumor-vessel density, that is, approximately 40% of those of the PBS and empty AAV treatments, respectively (each P<0.01); whereas there was no significant difference between the tumors treated with PBS and empty AAV (P>0.05) (FIG. 7A). Furthermore, within the tumors treated with rAAV-angiostatin, the median distance from an array of points to the nearest points labeled with anti-CD31 Ab was significantly larger than that observed with the tumors treated either with PBS or empty AAV (P<0.01 each) (FIG. 7B). Despite intensive research, the mechanism of antiangiogenic activity by angiostatin remains mostly unknown. Some studies have indicated that angiostatin can down-regulate vascular endothelial growth factor expression (Kirsch M. et al., 1998, supra; Joe Y. A. et al., 1999, supra). In the present study, rAAV-angiostatin had no significant effect on the expression of VEGF and this result was in line with one previous study (Sun et al., 2001, supra). However, tumoral VEGF expression, as detected by Western Blotting with a VEGF-specific antibody showed that VEGF expression slightly increased after rAAV-angiostatin treatment (FIG. 7C). This may be due to the increase in tumor hypoxia in the environment, by angiostatin-induced anti-angiogenesis, which may result in upregulation of VEGF expression via the pathway of hypoxia inducible factor that is a VEGF transcription factor. Similarly, Ding et al. (Intratumoral administration of endostatin plasmid inhibits vascular growth and perfusion in Mca-4 mammary carcinomas, Cancer Res. 2001; 61: 526-531) reported that intratumoral administration of endostatin caused a compensatory increase of in situ transcription of VEGF and VEGF receptor mRNAs.

6.9.5 rAAV-Angiostatin Increases Apoptosis of Tumor Cells but not of Hepatocytes

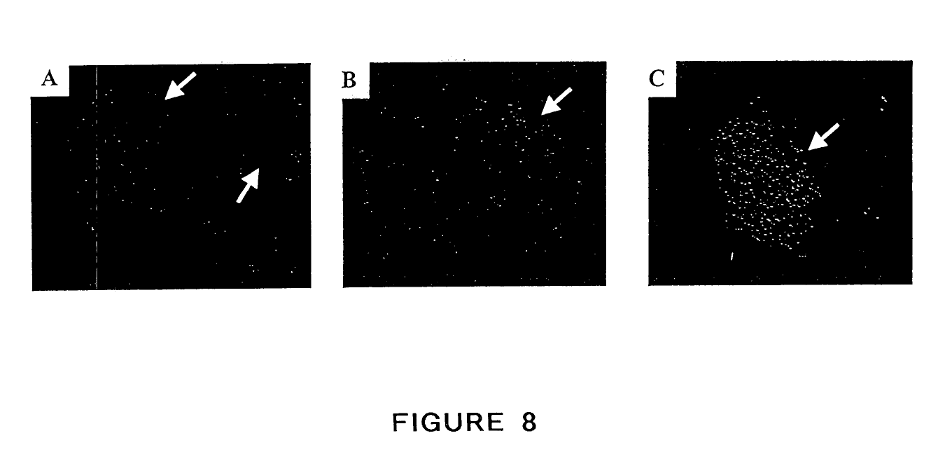

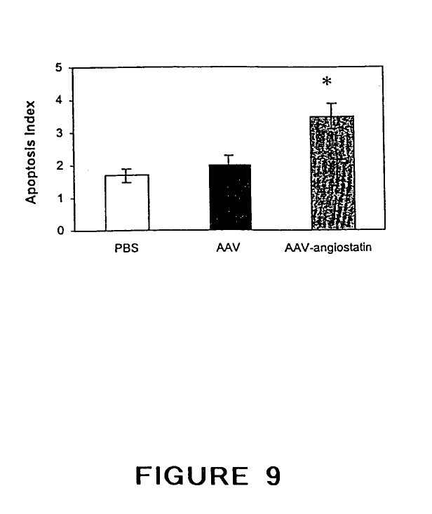

Since tumors can be starved for nutrients and oxygen as a result of rAAV-angiostatin treatment which prevents the formation of an adequate vascular network, a study was conducted to examine whether the tumors so treated underwent programmed death, by in situ labeling of fragmented DNA using the TUNEL method. A small number of apoptotic cells were detected in the tumors treated with PBS, or empty AAV (FIGS. 8A and 8B), while the number of apoptotic cells was doubled following rAAV-angiostatin treatment (FIG. 8C). The Apoptosis Index (AI) of the rAAV-angiostatin-treated group was significantly higher than that of the groups treated with PBS (P<0.001) and empty AAV groups (P<0.05), respectively (FIG. 9). There was no significant difference between the PBS- and empty AAV-treated groups. Furthermore, as shown in FIGS. 8A-8C, almost all the apoptotic cells were of the tumor cells and very few hepatocytes became apoptotic, indicating that the apoptotic effect of rAAV-angiostatin was highly selective for the tumor cells and did not affect normal liver cells. Lack of toxicity to normal liver cells clearly favors the clinical utilization of rAAV-angiostatin in treating the liver cancer.

6.10 Discussion

6.10.1 rAAV-Mediated Anti-Angiogenic Therapy is Advantageous Over Other Therapeutic Strategies

Localized intraportal delivery of rAAV expression vector into the liver, led to a persistent over-expression of exogenous angiogenesis inhibitor, angiostatin, in hepatocytes for up to 6 months and suppressed the growth of malignant liver metastasis.

Tumor growth and metastasis are dependent on the recruitment of a functional blood supply by a process known as tumor angiogenesis, and, indeed, the “angiogenic phenotype” correlates negatively with prognosis in many human solid tumors (Folkman J. Tumor angiogenesis: therapeutic implications. N Engl J Med. 1971; 17: 1-14). Antiangiogenic therapies devised so far target different steps of the angiogenic process, ranging from inhibition of expression of angiogenic molecules via overexpression of antiangiogenic factors, to direct targeting of tumor endothelial cells using endogenous angiogenic inhibitors or artificially constructed targeting ligands. In a controversial report, an intravenous administration of TNP-470, which is a typical angiogenesis inhibitor, suppressed the growth of primary tumors in a rat tumor model of Yoshida sarcoma, but increased the growth of metastatic foci in the lymph nodes (Hori K. et al. Increased growth and incidence of lymph node metastasises due to the angiogenesis inhibitor AGM-1470. Br J Cancer 1997; 75: 1730-1734). A low dosage or short-term administration of angiogenesis inhibitors may not be suitable for the treatment of metastatic cancer. Although a majority of preclinical and clinical anti-angiogenic therapies to date have been conducted with purified antiangiogenic factors, gene therapy appears to be more powerful than other forms of antiangiogenic therapy. Potential advantages of antiangiogenic gene therapy are sustained expression of the antiangiogenic factors and highly-localized delivery. Despite these advantages, the vector development for this form of therapy has been still in its infancy (Chen C. T. et al. Antiangiogenic gene therapy for cancer via systemic administration of adenoviral vectors expressing secretable endostatin. Human Gene Therapy 2000; 11: 1983-96; Feldman A. L. et al. Antiangiogenic gene therapy of cancer utilizing a recombinant adenovirus to elevate endostatin levels in mice. Cancer Res. 2000; 60: 1503-1506). Nonetheless, expression of antiangiogenic factors mediated by adenovirus-based vectors is limited by an effective host immune response and is also secondary due to the episomal nature of the vector.

6.10.2 Localized Delivery of AAV-Angiostatin Achieves Potential Therapeutic Efficacy in the Treatment of Liver Metastasis

Administration of a vector that constitutively expresses an antiangiogenic protein allows for the persistence of the protein in the circulation and has been shown to be more effective than the intermittent peaks of injected inhibitors in mice (Blezinger P. et al., 1999, supra). Adenoviruses that are designed to express angiostatin, endostatin, and neuropilin, respectively, were significantly less effective (Kuo C. J. et al. Comparative evaluation of the antitumor activity of antiangiogenic proteins delivered by gene transfer. Proc Natl Acad Sci USA 2001; 98: 4605-4610). However, when endostatin was transfected into tumor cells that were then implanted into mice, tumor growth was virtually completely inhibited (Feldman A. L. et al. Effect of retroviral endostatin gene transfer on subcutaneous and intraperitoneal growth of murine tumors. J Natl Cancer Inst. 2001; 93:1014-1020). The reason for the apparent difference in antitumor efficacy of endostatin between when it is free in the circulation (low efficacy) and when it is released locally in the tumor bed (high efficacy), is not clear. One possibility is that systemic gene therapy produces significantly higher plasma levels of endostatin than systemic protein therapy. If endostatin in the circulation follows a U-shaped curve of efficacy, then very high concentrations of the protein in the circulation might be less anti-angiogenic than lower doses. It has been previously reported that endostatin, when administered on a continuous intravenous schedule, resulted in 97% tumor regression in human BxPC3 pancreatic carcinoma when the dose reached 20 mg/kg/day and the serum level reached a steady state at approximately 250 ng/ml (Kisker O. et al. Continuous administration of endostatin by intraperitoneally implanted osmotic pump improves the efficacy and potency of therapy in a mouse xenograft tumor model. Cancer Res. 2001; 61: 7669-7674). However, when a very high dose of endostatin was administered at 400 mg/kg/day, there was only a 49% inhibition of tumor growth (Kerbel R. et al. Clinical translation of angiogenesis inhibitors. Nature Reviews/cancer 2002; 2: 727-739). Although these doses are in far excess of what a patient would receive, at least for systemic therapy, serum levels of endostatin may need to be carefully adjusted to generate blood levels in a certain range (Shi, W et al., 2002, Adeno-associated virus-mediated gene transfer of endostatin inhibits angiogenesis and tumor growth in vivo, Cancer Gene Ther. 9:513-521; Calvo A. et al. Adenovirus-mediated endostatin delivery results in inhibition of mammary gland tumor growth in C3 (1)/SV40 T-antigen transgenic mice. Cancer Res. 2002; 62: 3934-3938; Indraccolo S. et al. Differential effects of angiostatin, endostatin and interferon-i gene transfer on in vivo growth of human breast cancer cells. Gene Ther. 2002; 9: 867-878). However, the level of expressed protein in systemic circulation is not necessarily equal to its localized level inside tumors, let alone to reaching the levels in a narrow range. Localized vector delivery has been used to achieve or increase transgene expression in tumors in different gene therapy settings (Ju D. W. et al. Intratumoral injection of GM-CSF gene encoded recombinant vaccinia virus elicits potent antitumor response in a murine melanoma model. Cancer Gene Ther. 1997; 4: 139-144; Bass C. et al. Recombinant adenovirus-mediated gene transfer to genitourinary epithelium in vitro and in vivo. Cancer Gene Ther. 1995; 2: 97-104; de Roos W. K. et al. Isolated-organ perfusion for local gene delivery: efficient adenovirus-mediated gene transfer into the liver. Gene Ther. 1997; 4: 55-62; Lee S. S. et al. Intravesical gene therapy: in vivo gene transfer using recombinant vaccinia virus vectors. Cancer Res. 1994; 54: 3325-3328).

6.10.3 AAV-Angiostatin has the Ability to Induce Tumor Apoptosis Besides its Anti-Angiogenic Function

The mechanism of induction of tumor cell apoptosis by rAAV-angiostatin is unclear, though some studies have demonstrated that angiostatin-mediated inhibition of angiogenesis results in increased tumor cell apoptosis with no direct effect on the rate of tumor cell proliferation (Joe Y. A. et al., 1999, supra; Tanaka T. et al., 1998, supra; Griscelli F. et al., 1998, supra). The inhibition of neovascularization by angiostatin may restrict the supply of tumor cell-survival factors that are provided by endothelial cells and/or the circulation. Angiostatin has been also shown to induce apoptosis in endothelial cells that are critical for the formation of new blood vessels (Clasesson-Welsh L. et al. Angiostatin induces endothelial cell apoptosis and activation of focal adhesion kinase independently of the integrin-binding motif RGD. Proc Natl Acad Sci USA 1998; 95: 5579-5583; Lucas R. et al., Multiple forms of angiostatin induce apoptosis in endothelial cells. Blood 1998; 92: 4730-4741). The mechanism by which rAAV-angiostatin mediates tumor cell apoptosis may consist of cutting off the delivery of oxygen and nutrients. Thus, angiostatin may induce apoptosis in endothelial cells of microvessels which support the tumor cells, which, in turn, undergo apoptosis. Several studies indicate that angiogenesis inhibitors can induce tumor-cell apoptosis by decreasing levels of endothelial cell-derived paracrine factors that promote cell survival. At least 20 of these proteins, such as platelet derived growth factor (PDGF), IL-6 and heparin-binding epithelial growth factor (HB-EGF), among others, have been reported to be produced by endothelial cells (Rak J. et al. Consequences of angiogenesis for tumor progression, metastasis and cancer therapy. Anti-Cancer Drugs 1995; 6: 3-18). The decrease in production of paracrine factors is due, in part, to the inhibition of endothelial-cell proliferation (Dixelius J. et al. Endostatin-induced tyrosine kinase signaling through the Shb adaptor protein regulates endothelial cell apoptosis. Blood 2000; 95: 3403-3411). It is unclear whether angiogenesis inhibitors also directly decrease the production of paracrine factors by the endothelial cells.

6.10.4 AAV-Mediated Anti-Angiogenic Therapy is Useful for the Prevention and Treatment of Metastatic Liver Cancer

The present invention offers a useful clinical application of anti-angiogenic therapy for metastatic liver cancer. Removal of the primary tumors by surgery (O'Reilly M. S. et al., 1994, supra) or irradiation (Camphausen K. et al. Radiation therapy to a primary tumor accelerates metastatic growth in mice. Cancer Res. 2001; 61: 2207-2211) often results in the vascularization and rapid growth of disseminated microscopic remote tumors. The phenomenon called “concomitant resistance” can now be explained by the ability of one tumor to inhibit angiogenesis in the other (O'Reilly M. S. et al., 1994, supra). Certain tumors produce enzymes that activate angiogenesis inhibitors, such as angiostatin (O'Reilly M. S. et al., 1994, supra; Camphausen, K. et al., 2001, supra), endostatin (O'Reilly M. S. et al. Endostatin: an endogenous inhibitor of angiogenesis and tumor growth. Cell 1997; 88: 277-285; Wen W. et al. The generation of endostatin is mediated by elastase. Cancer Res. 1999; 59: 6052-6056; Felbor U. et al. Secreted cathepsin L generates endostatin from Collagen XVIII. EMBO J 2000; 19: 1187-1194) and anti-angiogenic anti-thrombin (O'Reilly M. S. et al. Antiangiogenic activity of the cleaved conformation of the serpin antithrombin. Science 1999; 285: 1926-1928; Kisker O. et al. Generation of multiple angiogenesis inhibitors by human pancreatic cancer. Cancer Res. 2001; 61:7298-7304), which in turn prevent the growth of remote tumors (O'Reilly M. S. et al., 1997, supra; Wen W. et al., 1999, supra).

Chemotherapy is the most common method of preventing and treating these microscopic disseminated metastatic tumors. However, its toxic and immune suppressing features devalue its clinical application. Antiangiogenic therapy is generally less toxic and less likely to induce acquired drug resistance. Thus, angiogenesis inhibitors can be used as a prophylactic measure for patients who have a high risk of cancer or as a therapy for a recurrence of cancer after complete surgical resection of primary tumors. An experimental study of spontaneous carcinogen-induced breast cancer in rats has revealed that endostatin prevented the onset of breast cancer and also prolonged the survival of the treated rats, compared with untreated controls (Choyke P. L. et al. Special techniques for imaging blood flow to tumors. Cancer J 2002; 8: 109-118). However, to achieve the preventive results, the anti-angiogenic reagents have to be delivered for a long time course and at high concentrations.

### 7. EXAMPLE 2

7.1 Methods

7.1.1 Generation of AAV-Angiostatin and AAV-B7.1

The cytomegalovirus (CMV) enhancer/chicken beta-actin promoter, reporter gene, a 1.4-kb cDNA fragment encoding full length of mouse angiostatin consisting of the signal peptide and the first four kringle regions of mouse plasminogen, or a 1.2 kb cDNA fragment encoding fill-length mouse B7.1, and poly A sequences were inserted between the inverted terminal repeats (ITRs) using appropriate restriction enzymes (see Xu L. et al. CMV-beta-actin promoter directs higher expression from an adeno-associated viral vector in the liver than the cytomegalovirus or elongation factor 1 alpha promoter and results in therapeutic levels of human factor X in mice. Hum Gene Ther. 2001; 12: 563-7). A woodchuck hepatitis B virus post-transcriptional regulatory element (WPRE) was also inserted into this construct to boost expression levels (Donello J. et al. Woodchuck hepatitis virus contains a tripartite posttranscriptional regulatory element. J Virol. 1998; 72: 5085-5092; Xu R. et al. Quantitative comparison of expression with adeno-associated virus (AAV-2) brain-specific gene cassettes. Gene Ther. 2001; 8: 1323-32). Plasmids were prepared using Qiagen plasmid purification kits.

AAV particles were generated by a three plasmid, helper-virus free packaging method (Xiao W. et al. Route of administration determines induction of T cell independent humoral response to adeno-associated virus vectors. Mol Ther. 2000; 1: 323-9) with slight modification. AAV-angiostatin and the helper pFdH22 were transfected into 293 cells using calcium phosphate precipitation. Cells were harvested 70 hours after transfection and lysed by incubation with 0.5% deoxycholate in the presence of 50 units/ml Benzonase® (Sigma) for 30 min at 37° C. After centrifugation at 5,000×g, they were filtered with a 0.45 μm Acrodisc® syringe filter to remove any particular matter prior to fractionation on a heparin column. The AAV particles were isolated by heparin affinity column chromatography. Peak virus fraction was dialyzed against 100 mM NaCl, 1 mM MgCl₂ and 20 mM sodium mono- and di-basic phosphate, pH 7.4.

A portion of the samples was subjected to quantitative PCR analysis using the AB Applied Biosystem, to quantify the genomic titer. The PCR Taqman® assay was a modified dot-blot protocol whereby AAV was serially diluted and sequentially digested with DNase I and Proteinase K. Viral DNA was extracted twice with phenol-chloroform to remove proteins, and then precipitated with 2,5 equivalent volumes of ethanol. A standard amplification curve was set up at a range from 102 to 107 copies and the amplification curve corresponding to each initial template copy number was obtained. Viral particles were reconfirmed using a commercial analysis kit (Progen, Germany). The viral vector was stored at −80° C. prior to animal experiments.

7.1.2 Mice, Cell Lines and Antibodies

Male C57BL/6 mice (H-2b), 6-8 weeks old, were obtained from the Laboratory Animal Unit of University of Hong Kong. The syngeneic (H-2b) EL-4 thymic lymphoma cell line was purchased from the American Type Culture Collection (Rockville, Md., USA). It was cultured at 37° C. in Dulbecco's Modified Eagles Medium (DMEM) (Gibco BRL, Grand Island, N.Y., USA), supplemented with 10% fetal calf serum (FCS), 50 U/ml penicillin/streptomycin, 2 mM L-glutamine, and 1 mM pyruvate. The anti-angiostatin monoclonal antibody (mAb) and anti-B7.1 mAb were purchased from Calbiochem-Novabiochem Corporation (Boston, Mass., USA) and BD Pharmingen (San Diego, Calif., USA), respectively.

7.1.3 Transfection of EL-4 Cells, and Analysis of Transgene Expression

Primary EL-4 cells (5×10⁵/well in 96-well plates) were incubated in a total volume of 50 μl of DMEM supplemented with 10% FCS and infectious AAV was added resulting in an MOL between 1 and 500. Cells were harvest at 0.5, 1, 2, 6, 12, 24, 48 hours. After being fixed with 4% paraformaldehyde solution, cells were blocked with 3% bovine serum albumin (BSA), and incubated with anti-B7.1 antibodies (Abs). They were then incubated with fluorescein-isothiocyanate (FITC)-conjugate secondary antibodies, and observed by fluorescence microscopy. Cells transfected with empty AAV vector alone served as controls.

7.1.4 Flow Cytometry

After AAV transduction, EL-4 tumor cells were harvested, purified by Ficoll density gradient centrifugation, and washed. Cells were incubated with specific Abs for 30 min in phosphate buffered saline (PBS), 4% FCS, 0.1% sodium azide, 20 mM HEPS(N-2-hydroxyethylpiperazine-N′-2 ethanesulfonic acid), and 5 mM ethylenediaminetetraacetic acid (EDTA), pH 7.3, on ice and washed. Nonspecific binding was controlled by incubation with an isotypic control rat IgG1 mAb (BD Pharmingen). Cells transfected with empty AAV vector alone served as controls. The level of expression of the transgene was assessed by FACScan analysis. Cells were then used as cytotoxic T lymphocyte (CTL) targets as described below and for animal experiments.

7.1.5 Animal Experiments

All surgical procedures and care administered to the animals were approved by the Ethics Committee of the University of Hong Kong and performed according to institutional guidelines. Animals were randomly assigned to treatment. Each group contained 10 mice. The disseminated tumor models consistently yielded tumors in at least 90-95% animals. Equal numbers of parental EL-4 cells and equal numbers of empty AAV virus particles served as controls.

7.1.5.1 Immunization of Mice

C57BL mice were anesthetized with 10% ketamine/xylazine solution by intraperitoneal injection, and their abdomens were prepared with Betadine solution. A right subcostal incision was used to open the abdominal cavity. After the hilar of the liver was surgically exposed, 2×10⁵ AAV-B7.1 transfected EL-4 tumor cells were slowly injected into the portal vein with a 30-gauge needle, and pressure was applied with a sterile cotton tip applicator until the injection site was haemostatic. Homeostasis was performed and the abdominal cavity was closed. The mice were laparotomized under anesthetization to observe tumors on the surface of livers 4 weeks later. The mice with visible tumors were killed, and their livers excised. The livers were then frozen and cryostated to prepare transverse 10 μm sections, which were made at 5 different levels to cover the entire liver. The sections were mounted and stained with haematoxylin and eosin. The entire liver and tumor areas were measured and examined under microscopy with a Sigma Scan program. The relative areas occupied by the tumors were calculated in accordance with the following formula: total tumor areas/liver area×100. The mice without visible tumors on the surface of livers were used for the following experiments.

7.1.5.2 Challenge of the Vaccinated Mice With Parental Tumor Cells

The mice without visible tumors on the surface of livers from the experiments above were intraportally injected with 2×10⁵ or 2×10⁶ parental EL-4 tumor cells to detect whether systemic anti-tumor immunity had been generated. Four (4) weeks later the mice were killed and hepatomized. The relative areas occupied by tumors in the livers were analyzed as above.

7.1.5.3 AAV-Angiostatin Therapy to Combat Disseminated Liver Cancers in Vaccinated Mice

Mice vaccinated with AAV-B7.1 transfected EL-4 tumor cells and found to be free of liver tumors were intraportally injected with 2×10⁶ parental EL-4 tumor cells with a 30-gauge needle, followed by intraportal transfusion of 3×10¹¹ particles of AAV-angiostatin. Pressure was applied with a sterile cotton tip applicator until the injection site was hemostatic. Homeostasis was performed and the abdominal cavity was closed. Unvaccinated mice and empty AAV virus were used as controls. Four weeks after the operation, the mice were killed, and their livers excised. The relative areas occupied by tumors in the livers were analyzed as above.

7.1.5.4 Survival Studies

Mice vaccinated with AAV-B7.1 transfected EL-4 tumor cells and found to be free of liver tumors were intraportally injected with 2×10⁶ parental EL-4 tumor cells with a 30-gauge needle, followed by intraportal transfusion of 3×10¹¹ particles of AAV-angiostatin. Pressure was applied with a sterile cotton tip applicator until the injection site was hemostatic. Homeostasis was performed and the abdominal cavity was closed. Unvaccinated mice and empty AAV virus were used as controls. The animals were weighed thrice weekly and assessed. Moribund mice were euthanized according to pre-established criteria, namely the presence of two or more of the following premorbid conditions: (1) gross ascites, (2) palpable tumor burden greater than 2 cm, (3) dehydration, (4) lethargy, (5) emaciation, and (5) weight loss greater than 20% of initial body weight.

7.1.6 Immunohistochemistry of Tissue Sections

Cryosections (10 μm thickness) prepared from livers following intraportal delivery of therapeutic agents were incubated overnight with specific Abs. They were subsequently incubated for 30 min with appropriate secondary antibodies (VECTASTAIN® Universal Quick kit, Vector Laboratories, Burlingame, Calif.), and developed with Sigma FAST™ DAB (3,3′-diaminobenzidine tetrahydrochloride) and CoCl₂ enhancer tablets (Sigma). Sections were counterstained with Mayer's hematoxylin.

7.1.7 Western Blotting Analysis

In vitro transfected cells were harvested, or tissues from mice were excised and minced and homogenized in protein lysate buffer. Debris was removed by centrifugation at 10,000×g for 10 min at 4° C. Lysates from each group of mice were pooled, and protein content determined. Protein samples (100 μg) were resolved on 10% polyacrylamide SDS gels, and electrophoretically transferred to nitrocellulose Hybond™-C extra membranes (Amersham Life Science, England). After the membranes were blocked with 5% BSA, blots were incubated with specific primary Abs, followed by horseradish peroxidase-conjugated secondary antibodies, and developed by enhanced chemiluminescence (Amersham International plc, England) and exposure to X-Ray film. Band density was quantified using Sigma Scan Program.

7.1.8 Cytotoxicity Assays

Splenocytes were harvested from mice vaccinated with AAV-B7.1 transfected EL-4 tumor cells and found to be free of liver tumors, and incubated at 37° C. with EL-4 target cells in graded E:T ratios in 96-well round-bottom plates. After a 4 hour incubation, 50 μl of supernatant was collected, and lysis was measured using the Cyto Tox 96 Assay kit (Promega, Madison, Wis., USA). Background controls for non-specific target and effector cell lysis were included. After background subtraction, the percentage of cell lysis was calculated using the formula: 100×(experimental-spontaneous effector-target spontaneous target/maximum target-spontaneous target).

7.1.9 In Situ Hybridization

Liver sections were fixed for 7 min in 4% formaldehyde, washed in PBS for 3 min, and then in 2×SSC for 10 min. Dehydrated sections were hybridized overnight at 60° C. with probe solution according to an established in situ hybridization protocol (Ambion, Austin, Tex., USA). Slides were washed with 4×SSC, and incubated in RNase digestion solution at 37° C. for 30 min, followed by washing with decreasing concentrations of SSC at room temperature for periods of 5 min with gentle agitation. Slides were dehydrated with an increasing concentration of ethanol, and hybridization performed using a VECTASTAIN® ABC kit and an Alkaline Phosphatase chromogen kit (BCIP/NBT).

7.2 Results

7.2.1 In Vitro Fast and Efficient Transfection of EL-4 Tumor Cells With AAV-B7.1

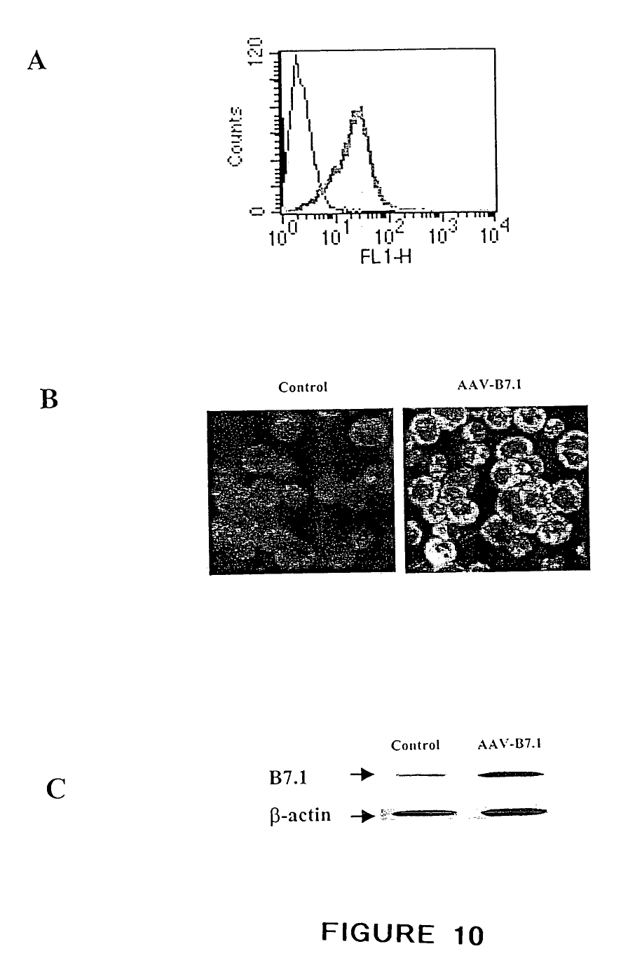

The efficiency of transfection of parental EL-4 tumor cells by AAV-B7.1 viruses was analyzed by measuring the expression of B7.1 on the cell surface by flow cytometry (FIG. 10A), and confirmed by immunohistochemistry (FIG. 10B) and Western blotting analysis (FIG. 10C). EL-4 cells transfected with AAV-B7.1 viruses expressed higher levels of B7.1 compared to untransfected parental EL-4 cells. After 6 hours of incubation, over 80% of the EL-4 tumor cells transfected with AAV-B7.1 expressed increased levels of B7.1. Transfectants were then used for the following experiments.

7.2.2 Persistent Expression of Angiostatin in the Liver After AAV-Portal Vein Transfusion

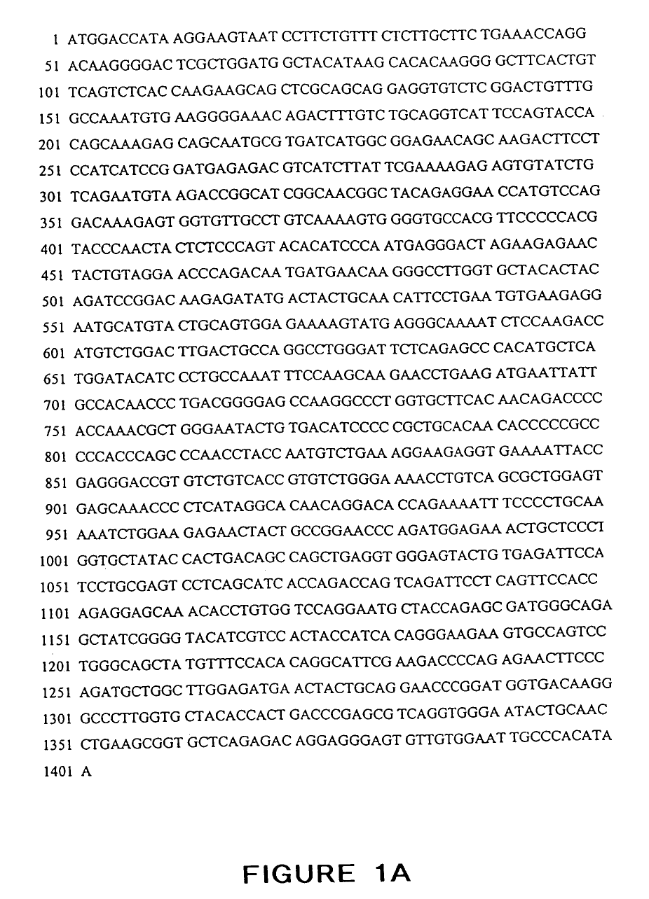

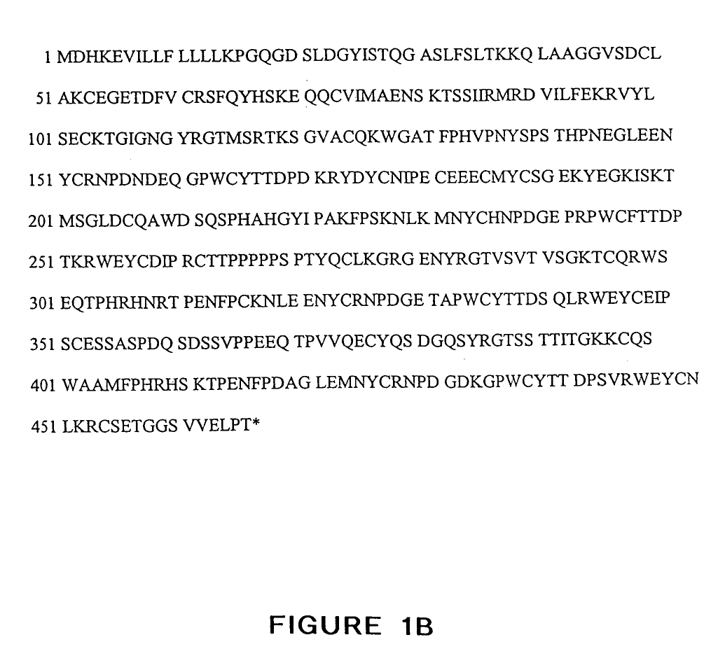

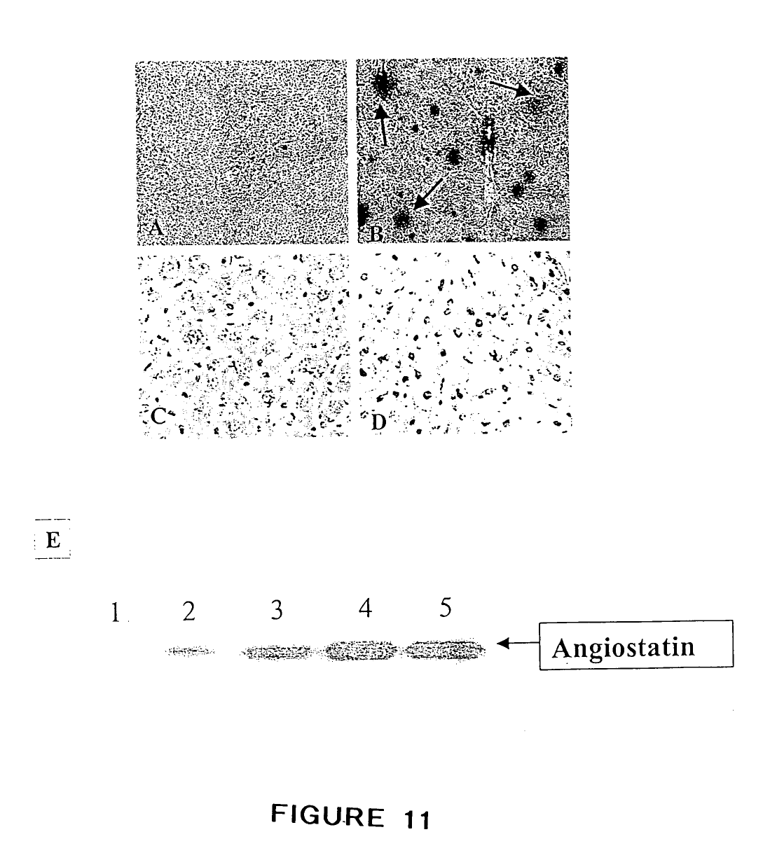

We previously reported that injection of a recombinant AAV-angiostatin vector via a portal vein leads to long-term exogenous gene expression in the liver (see U.S. Provisional Application No. 60/438,449, filed Jan. 7, 2003; and Xu R. et al. Long-term expression of angiostatin suppresses liver metastatic cancer in mice. Hepatology. 2003; 37(6): 1451-60, which are incorporated herein by reference in their entireties). In the latter study, angiostatin protein was overexpressed in hepatocytes 14 days following intraportal injection of AAV-angiostatin, and increased levels persisted for at least 180 days. Similar results were achieved in the present study, where liver samples were collected at 2, 14, 60, and 180 days after intraportal injection of AAV-angiostatin. Empty AAV was used as a control. Angiostatin expression in the liver was confirmed by immunohistochemistry, in situ hybridization and Western blotting. As shown in FIG. 11, angiostatin was clearly overexpressed in hepatocytes 14 days following gene transfer (B), compared to low endogenous levels in livers treated with empty vector control (A), as detected by in situ hybridization of liver sections with a DIG-labeled antisense WPRE. Overexpression of angiostatin following intraportal injection of AAV-angiostatin was further confirmed by immunohistochemistry of liver sections (FIG. 11D, compared to FIG. 11C). Western blot analysis of liver homogenates indicated that transgenic angiostatin expressed in hepatocytes rose rapidly to a high level in two weeks, increased to a peak level in two months, and then was stably expressed at a constant level until at least 6 months after injection of AAV-angiostatin (FIG. 1I E).

7.2.3 AAV-B7.1 Transfection Stimulates Tumor-Specific Cytolytic T Cell Activity in a Intraportal Transfusion Mouse Model

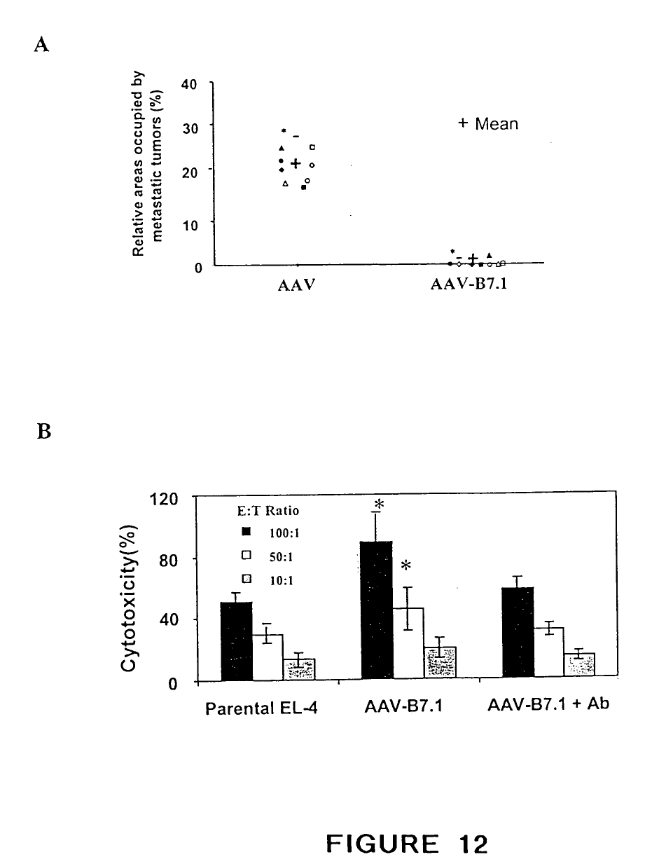

To analyze the formation and growth of disseminated hepatic metastatic tumors, 2×10⁵ EL-4 cells that had been transfected with AAV-B7.1 were intraportally injected into the livers of mice (n=10). Tumour formation and growth was compared with intraportal injection of a similar number of EL-4 cells transfected with empty AAV into control mice. Four weeks later, all the mice underwent laparotomy, and mice with visible tumors were hepatomized. Livers were sectioned, and relative areas occupied by tumors were measured with a Sigma Scan program as illustrated in FIG. 12A. The mean relative areas were 22.9% and 3.2% after treatment with EL-4 cells transfected with either AAV-B7.1 or empty AAV, respectively. Vaccination with AAV-B7.1-transfected EL-4 cells led to statistically significant (P<0.001) reductions (86%) in the relative areas occupied by tumors. Furthermore, 60% of the mice were free of liver tumors. The mice without visible tumors on the surface of livers were used for the following experiments.

To assess whether expression of B7.1 by EL-4 transfectants facilitates tumor cell lysis by anti-tumor CTL, an in vitro CTL killing assay was devised where splenocytes from tumor-challenged mice cured by vaccination with AAV-B7.1 transfectants were mixed with EL-4 cells that had either been transfected with AAV-B7.1 or empty AAV. At an effector to target ratio of 50:1, anti-tumor CTL showed highly significant (P<0.01) killing of tumor cells transfected with AAV-B7.1 compared to killing of EL-4 cells transfected with empty AAV. Thus, exogenous B7.1 facilitates killing by anti-tumor CTL; an effect that could be abrogated by anti-B7.1 antibodies (FIG. 12B).

7.2.4 Memorized Anti-Tumor Immunity Induced by AAV-B7.1 is Tumor-Specific and Protects Against a Subsequent Tumor Challenge

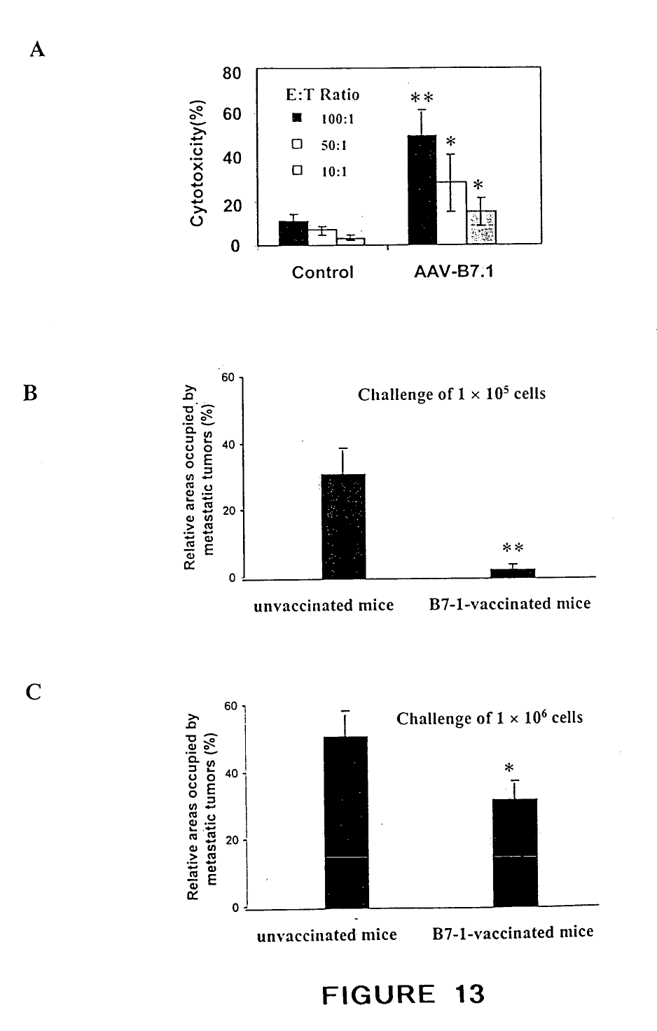

The anti-tumor CTL activity displayed by splenocytes from cured mice free of tumors, that had been intraportally injected 28 days earlier with AAV-B7.1 transfected EL-4 cells, was significantly (P<0.01) augmented versus splenocytes from mice that had received empty AAV transfected EL-4 cells (FIG. 13A). The mice, which had been cured of their tumors by intraportal injection of AAV-B7.1 transfected EL-4 cells, were rechallenged by intraportal injection of 2×10⁵ parental EL-4 tumor cells. Tumors reappeared in only one of ten mice, indicating that systemic anti-tumor immune memory activity had been established. In contrast, disseminated hepatic tumors appeared in 100% of the unvaccinated mice with significantly large areas (up to 38%) occupied by tumors (FIG. 13B).

7.2.5 The Anti-Tumor Immunity Induced by AAV-B7.1 Failed to Protect Against a Challenge With a Heavy Burden of Parental EL-4 Tumor Cells

The mice, which had been cured of tumors after intraportal injection of AAV-B7.1 transfected EL-4 cells, were rechallenged by intraportal injection of a much larger number (2×10⁶) of parental EL-4 tumor cells. Tumors that had metastasized to the liver were observed in all the mice, though the average relative areas occupied by tumors were significantly smaller (P<0.01) in vaccinated mice than in unvaccinated mice (FIG. 13C).

7.2.6 AAV-Angiostatin Enhances the Therapeutic Efficacy of the AAV-B7.1 Vaccine

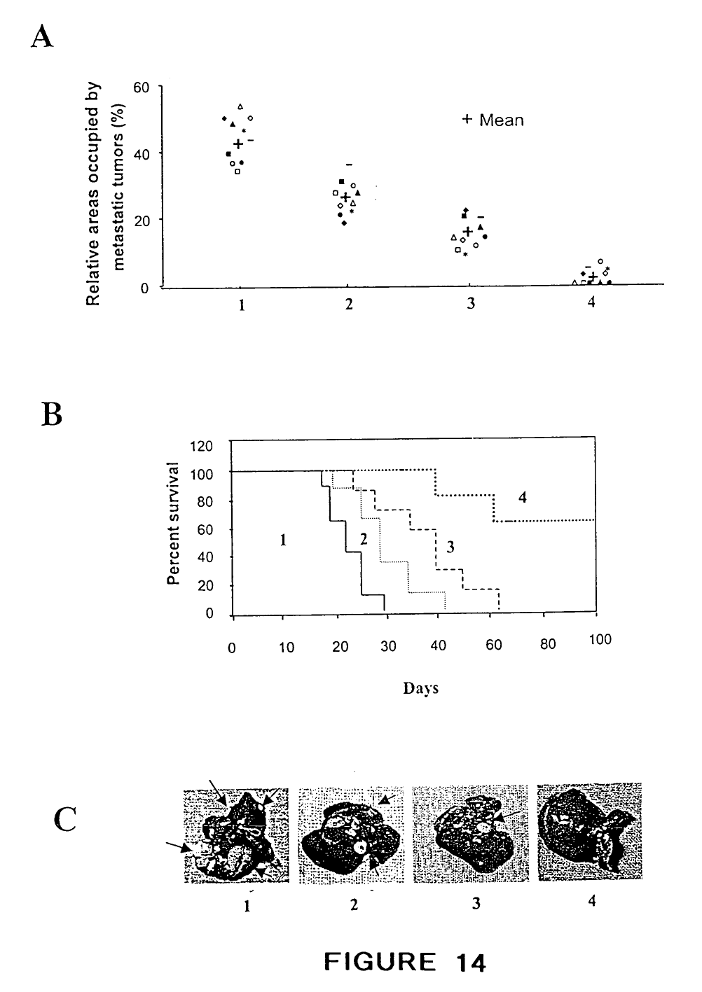

AAV-B7.1 transfected EL-4 tumor cells (2×10⁵) were intraportally injected into the livers of mice. Four weeks later, these vaccinated mice underwent laparotomy to observe visible liver tumors. The mice with visible liver tumors were excluded from the experiments. All the mice without tumors were intraportally injected with 2×10⁶ EL-4 parental cells, followed by intraportal injection of either empty AAV (n=10), or AAV-angiostatin (n=10). Unvaccinated mice used as controls were intraportally injected with 2×10⁶ parental EL-4 cells, followed by either empty AAV (n=10), or AAV-angiostatin (n=10). The mice were sacrificed 4 weeks later, hepatectomized, and the livers transversely sectioned. The relative areas occupied by tumors in the livers are illustrated in FIG. 14A. The mean relative areas of tumors in unvaccinated mice receiving either empty AAV or AAV-angiostatin were 42.3% and 17.7%, respectively. Thus, AAV-angiostatin significantly suppressed the growth of tumors that had metastasized to the liver by 56%, in accord with our previous report (Xu R. et al. Long-term expression of angiostatin suppresses liver metastatic cancer in mice. Hepatology. 2003; 37(6): 1451-60). Vaccination with AAV-B7.1 transfected EL-4 cells also significantly suppressed the growth of tumors by 38% such that 26.2% of the liver was occupied by tumors, compared with 42.3% of the liver in the unvaccinated mice. The mean relative area occupied by liver tumors in mice vaccinated with AAV-B7.1 transfected EL-4 cells and treated with AAV-angiostatin was only 5.6%, and only 50% (5/10) mice had visible liver tumors. The reduction in the relative areas occupied by liver tumors was decreased by 87% compared to unvaccinated mice treated with empty AAV, by 79% compared to mice vaccinated with AAV-B7.1 transfected EL-4 cells and treated with empty AAV, and by 68% compared to unvaccinated mice treated with AAV-angiostatin.

7.2.7 AAV-B7.1 and AAV-Angiostatin Synergize in Improving the Survival Rate of Mice Bearing Liver Metastases

We further investigated whether the synergy obtained by vaccination with AAV-B7.1 transfected EL-4 cells followed by AAV-angiostatin therapy would offer a survival benefit for mice. C57BL/6 mice were intraportally injected with 2×10⁵ AAV-B7.1 transfected EL-4 tumor cells. Four weeks later, all the mice underwent laparotomy. The mice with visible tumors in the livers were excluded from the experiments. All the mice without tumors were intraportally injected with 2×10⁶ EL-4 parental cells, followed by intraportal injection of either 3×10¹¹ particles of empty AAV (n=10), or 3×10¹¹ particles of AAV-angiostatin (n=10). Unvaccinated mice used as controls were intraportally injected with 2×10⁶ parental EL-4 cells, followed by intraportal injection of either 3×10¹¹ particles of empty AAV (n=10), or 3×10¹¹ particles of AAV-angiostatin (n=10). Both vaccination with AAV-B7.1 transfected EL-4 cells and AAV-angiostatin therapy resulted in significant improvement in the survival of mice, compared to unvaccinated mice treated with empty AAV. Furthermore, the combinational therapy led to a statistically longer survival rate. Six of ten mice in the combined therapy group survived for more than 100 days after tumor cell inoculation (FIG. 14B). Median survival times for mice vaccinated with AAV-B7.1 transfected EL-4 cells and treated with empty AAV, or for unvaccinated mice treated with AAV-angiostatin was 33 days and 42 days, respectively, which are significantly (P<0.05 or P<0.01, respectively) different from the median survival time of 25 days for unvaccinated control mice treated with empty AAV (FIG. 14B).

7.3 Discussion

Many of the most common cancers metastasize to the liver. A majority of patients succumb to colorectal and breast cancers with multiple metastases predominantly in the liver. A clinical impact requires a systemic or regional therapy directed at all the liver metastases (Tada H. et al. Systemic IFN-β gene therapy results in long-term survival in mice with established colorectal liver metastases. J Clin Invest. 2001; 108: 83-95). The impetus for the present study stemmed from a previous report in which we demonstrated that the immune resistance of large tumors can be overcome by combining B7.1-mediated immunotherapy with a concerted attack on the tumor vasculature delivered by gene transfer of angiostatin (Sun X. et al. Cancer Gene Ther. 2001; 8: 719-727), and another report where we showed that intraportal transfusion of a recombinant AAV vector encoding mouse angiostatin leads to long term and persistent expression of angiostatin in livers and significantly suppresses metastatic lver tumors (Xu R. supra.). However, anti-angiogenic therapy using AAV-angiostatin could not eradicate metastatic liver tumors, presumably because while anti-angiogenic proteins are effective at inducing tumor regression, they are not directly tumoricidal, and hence tumor regrowth frequently reoccurs once treatment is suspended. To expand the scope of cancer gene therapy in combination with immunotherapy and anti-angiogenic therapy, we have employed AAV technology to deliver both angiostatin and the costimulatory molecule B7.1.

The present study demonstrates for the first time that localized intraportal delivery of AAV-B7.1 transfected EL-4 cells induces memorized anti-tumor immunity, which renders vaccinated mice with the ability to resist to a challenge with parental EL-4 cells. Combinational intraportal transfusion of AAV-B7.1 transfected EL-4 cells and AAV-angiostatin was able to eradicate established liver metastatic tumors.

Since B7-dependent costimulatory signals play a central role in T cell activation, it has been proposed that the lack of immunogenicity of many tumor types could be due to the lack of B7 expression (Chen L. et al. Costimulation of antitumor immunity by the B7 counterreceptor for the T lymphocyte molecules CD28 and CTLA-4. Cell. 1992; 71: 1093-102; Baskar S. et al. Constitutive expression of B7 restores immunogenicity of tumor cells expressing truncated major histocompatibility complex class II molecules. Proc Natl Acad Sci USA. 1993; 90(12): 5687-90). Indeed, it was proved that transfection of B7.1 genes into different experimental mouse tumors greatly improved their immunogenicity (Chen L. et al. supra.; Baskar S. et al. supra.). Transfection of B7-1 and B7-2 into immunogenic tumor cells attributes-such cells with an ability to present their tumor antigens and to generate anti-tumor CTLs, leading to prevention of tumorigenesis when transfectants are injected into animals. In contrast, the immune system remains completely ignorant of the parental nontransfected tumor cells, which grow unchecked (Chen L. et al. Costimulation of antitumor immunity by the B7 counterreceptor for the T lymphocyte molecules CD28 and CTLA-4. Cell. 1992; 71: 1093-102; Townsend S. E. et al. Tumor rejection after direct costimulation of CD8+ T cells by B7-transfected melanoma cells. Science 1993; 259: 368-370; Baskar S. et al. Constitutive expression of B7 restores immunogenicity of tumor cells expressing truncated major histocompatibility complex class II molecules. Proc Natl Acad Sci USA. 1993; 90(12): 5687-90). Intratumoral gene transfer of mouse B7-1 and -2, which can eradicate already established tumors, has been shown to costimulate anti-tumor activity mediated by CD8+ T cells and NK cells, accompanied by augmented tumor-specific cytolytic T cell activity involving both the perforin and Fas-ligand pathways (Sun X. Cancer Gene Ther. 2001; 8: 719-727; Kanwar J. R. et al. Gene Therapy 1999; 6:1835-1844; Sun X. et al. Gene Ther 2001; 8: 638-645; Kanwar J. R. et al. Effect of surviving antagonists on the growth of established tumors and B7.1 immunogene therapy. J Natl Cancer Inst. 2001; 93: 1541-1552).

The AAV mediated transfection system used in the present study is advantageous as it could quickly transfect EL-4 tumor cells in vitro, thus transforming parental EL-4 cells into a vaccine, which could be used to immunize mice. The vaccinated mice resisted the challenge with parental EL-4 cells, indicating anti-tumor immunity was generated.

The key finding of the present study is that angiostatin and B7.1-immunotherapy synergize in causing the eradication of tumors that metastasize to the liver. In contrast, neither vaccination with AAV-B7.1 transfected EL-4 cells nor AAV-angiostatin monotherapy were effective in clearing tumors that metastasized to the liver. Mice cured by combination therapy and rechallenged with live parental EL-4 cells remained tumor-free for at least 2 months, indicating that potent systemic anti-tumor immunity had been generated.

Localized vector delivery has been used to specifically target transgene expression within tumors (Bass C. et al. Recombinant adenovirus-mediated gene transfer to genitourinary epithelium in vitro and in vivo. Cancer Gene Ther. 1995; 2: 97-104; de Roos W. K. et al. Isolated-organ perfusion for local gene delivery: efficient adenovirus-mediated gene transfer into the liver. Gene Ther. 1997; 4: 55-62; Lee S. S. et al. Intravesical gene therapy: in vivo gene transfer using recombinant vaccinia virus vectors. Cancer Res. 1994; 54: 3325-3328). Although systemic vector delivery may be the best option in many clinical settings, the unique anatomic features of the liver facilitate regional gene therapy approaches for unresectable hepatic metastases (de Roos et al. supra.). The advantages of localized vector delivery are obvious, as it can induce high level expression of transgenic proteins in situ to achieve effective anti-tumor activity, and reduce the possibility of side-effects compared to the systemic delivery.

The combinational gene therapy approach described herein using the costimulatory molecule B7.1 and the angiogenesis inhibitor angiostatin led to persistent over-expression of exogenous angiostatin in hepatocytes for up to 6 months, and suppressed the growth of lymphomas that had metastasized to the liver. The results have important implications for the treatment of cancers of the liver, which are most often intractable to treatment.

### 8. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

What is claimed:

1. A nucleic acid molecule comprising an adeno-associated viral vector, and a CAG promoter which is operably linked to a nucleic acid sequence encoding angiostatin, wherein the CAG promoter comprises a cytomegalovirus enhancer and beta-actin promoter.

2. The nucleic acid molecule of claim 1 further comprising a woodchuck hepatitis B virus post-transcriptional regulatory element.

3. A nucleic acid molecule comprising an adeno-associated viral vector and a CAG promoter which is operably linked to: (a) the nucleotide sequence of SEQ ID NO:1; or (b) a nucleotide sequence that encodes the amino acid sequence of SEQ ID NO:2, wherein the CAG promoter comprises a cytomegalovirus enhancer and beta-actin promoter.

4. The nucleic acid molecule of claim 3 further comprising a woodchuck hepatitis B virus post-transcriptional regulatory element.

5. A vector comprising the nucleic acid molecule of claim 1.

6. A host cell comprising the vector of claim 5.

7. A pharmaceutical composition comprising the nucleic acid molecule of claim 1, and a pharmaceutically acceptable carrier.

8. A nucleic acid molecule comprising an adeno-associated viral vector, and a CAG promoter which is operably linked to a nucleic acid sequence encoding B7.1, wherein the CAG promoter comprises a cytomegalovirus enhancer and beta-actin promoter.

9. The nucleic acid molecule of claim 8 further comprising a woodchuck hepatitis B virus post-transcriptional regulatory element.

10. A nucleic acid molecule comprising an adeno-associated viral vector and a CAG promoter which is operably linked to: (a) the nucleotide sequence of SEQ ID NO:3; or (b) a nucleotide sequence that encodes the amino acid sequence of SEQ ID NO:4, wherein the CAG promoter comprises a cytomegalovirus enhancer and beta-actin promoter.

11. The nucleic acid molecule of claim 10 further comprising a woodchuck hepatitis B virus post-transcriptional regulatory element.

12. A vector comprising the nucleic acid molecule of claim 8.

13. A host cell comprising the vector of claim 12.

14. A pharmaceutical composition comprising the nucleic acid molecule of claim 8, and a pharmaceutically acceptable carrier.

15. A method for the production of isolated or purified angiostatin protein, or a fragment, variant, or derivative thereof, said method comprising (i) growing the cell of claim 6 such that angiostatin protein is expressed; and (ii) isolating or purifying said angiostatin protein.

16. A method for the production of isolated or purified B7.1 protein, or a fragment, variant, or derivative thereof, said method comprising (i) growing the cell of claim 13 such that B7.1 protein is expressed; and (ii) isolating or purifying said B7.1 protein.

17. A method of treating or preventing cancer in a subject in need thereof, said method comprising administering to said subject a therapeutically effective amount of a nucleic acid molecule comprising an adeno-associated viral vector and a CAG promoter which is operably linked to a nucleic acid sequence encoding angiostatin, wherein the CAG promoter comprises a cytomegalovirus enhancer and beta-actin promoter.

18. A method of treating or preventing cancer in a subject in need thereof, said method comprising administering to said subject a therapeutically effective amount of a nucleic acid molecule comprising an adeno-associated viral vector and a CAG promoter which is operably linked to a nucleic acid sequence encoding B7.1, wherein the CAG promoter comprises a cytomegalovirus enhancer and beta-actin promoter.

19. A method of treating or preventing cancer in a subject in need thereof, said method comprising administering to said subject a therapeutically effective amount of a nucleic acid molecule comprising an adeno-associated viral vector and a CAG promoter which is operably linked to: (a) the nucleotide sequence of SEQ ID NO:1; or (b) a nucleotide sequence that encodes the amino acid sequence of SEQ ID NO:2, wherein the CAG promoter comprises a cytomegalovirus enhancer and beta-actin promoter.

20. A method of treating or preventing cancer in a subject in need thereof, said method comprising administering a prophylactically effective amount of a nucleic acid molecule comprising an adeno-associated viral vector and a CAG promoter which is operably linked to: (a) the nucleotide sequence of SEQ ID NO:3; or (b) a nucleotide sequence that encodes the amino acid sequence of SEQ ID NO:4, wherein the CAG promoter comprises a cytomegalovirus enhancer and beta-actin promoter.

21. The method of claim 17 or 18, wherein the nucleic acid molecule further comprises a woodchuck hepatitis B virus post-transcriptional regulatory element.

22. The method of claim 17 or 18, wherein said cancer is liver cancer.

23. The method of claim 22, wherein said liver cancer is metastatic.

24. The method of claim 17 or 18, wherein the nucleic acid molecule is administered via a portal vein.

25. The method of claim 17 or 18, wherein the nucleic acid molecule is administered by muscular injection.

26. A method of treating or preventing cancer in a subject in need thereof, said method comprising administering to said subject a therapeutically effective amount of:
(a) a first nucleic acid molecule comprising an adeno-associated viral vector and a CAG promoter which is operably linked to a nucleic acid sequence encoding angiostatin, wherein the CAG promoter comprises a cytomegalovirus enhancer and beta-actin promoter; and
(b) a second nucleic acid molecule comprising an adeno-associated viral vector and a CAG promoter which is operably linked to a nucleic acid sequence encoding B7.1, wherein the CAG promoter comprises a cytomegalovirus enhancer and beta-actin promoter.

27. A method of treating or preventing cancer in a subject in need thereof, said method comprising administering to said subject a therapeutically effective amount of:
(a) a first nucleic acid molecule comprising an adeno-associated viral vector and a CAG promoter which is operably linked to: (a) the nucleotide sequence of SEQ ID NO:1; or (b) a nucleotide sequence that encodes the amino acid sequence of SEQ ID NO:2, wherein the CAG promoter comprises a cytomegalovirus enhancer and beta-actin promoter; and
(b) a second nucleic acid molecule comprising an adeno-associated viral vector and a CAG promoter which is operably linked to: (a) the nucleotide sequence of SEQ ID NO:3; or (b) a nucleotide sequence that encodes the amino acid sequence of SEQ ID NO:4, wherein the CAG promoter comprises a cytomegalovirus enhancer and beta-actin promoter.

28. The method of claim 26, wherein the first nucleic acid molecule further comprises a woodchuck hepatitis B virus post-transcriptional regulatory element.

29. The method of claim 26, wherein the second nucleic acid molecule further comprises a woodchuck hepatitis B virus post-transcriptional regulatory element.

30. The method of claim 26, wherein said cancer is liver cancer.

31. The method of claim 30, wherein said liver cancer is metastatic.

32. The method of claim 26, wherein the first nucleic acid molecule and second nucleic acid molecule are administered via a portal vein.

33. The method of claim 26, wherein the first nucleic acid molecule and second nucleic acid molecule are administered by muscular injection.

34. The method of claim 26, wherein the first nucleic acid molecule and the second nucleic acid molecule are administered sequentially.

35. The method of claim 26, wherein the first nucleic acid molecule and the second nucleic acid molecule are administered simultaneously.

36. A nucleic acid molecule comprising an adeno-associated viral vector, and a CAG promoter which is operably linked to a first polynucleotide comprising a first nucleic acid sequence encoding angiostatin, and a second polynucleotide comprising a second nucleic acid sequence encoding B7.1, wherein the CAG promoter comprises a cytomegalovirus enhancer and beta-actin promoter.

37. The nucleic acid molecule of claim 36 further comprising a woodchuck hepatitis B virus post-transcriptional regulatory element.

38. A nucleic acid molecule comprising an adeno-associated viral vector and a CAG promoter which is operably linked to: (a) a first polynucleotide comprising (i) the nucleotide sequence of SEQ ID NO:1, or (ii) a nucleotide sequence that encodes the amino acid sequence of SEQ ID NO:2; and (b) a second polynucleotide comprising (i) the nucleotide sequence of SEQ ID NO:3, or (ii) a nucleotide sequence that encodes the amino acid sequence of SEQ ID NO:4, wherein the CAG promoter comprises a cytomegalovirus enhancer and beta-actin promoter

39. The nucleic acid molecule of claim 38 further comprising a woodchuck hepatitis B virus post-transcriptional regulatory element.

40. A vector comprising the nucleic acid molecule of claim 36.

41. A host cell comprising the vector of claim 40.

42. A pharmaceutical composition comprising the nucleic acid molecule of claim 36, and a pharmaceutically acceptable carrier.

43. A method for the production of isolated or purified B7.1 protein and angiostatin, or a fragment, variant, or derivative thereof, said method comprising (i) growing the cell of claim 41 such that B7.1 protein and angiostatin are expressed; and (ii) isolating or purifying said B7.1 protein and angiostatin.

44. A method of treating or preventing cancer in a subject in need thereof, said method comprising administering to said subject a therapeutically effective amount of a nucleic acid molecule comprising an adeno-associated viral vector, and a CAG promoter which is operably linked to a first polynucleotide comprising a first nucleic acid sequence encoding angiostatin, and a second polynucleotide comprising a second nucleic acid sequence encoding B7.1, wherein the CAG promoter comprises a cytomegalovirus enhancer and beta-actin promoter.

45. A method of treating or preventing cancer in a subject in need thereof, said method comprising administering to said subject a therapeutically effective amount of a nucleic acid molecule comprising an adeno-associated viral vector and a CAG promoter which is operably linked to: (a) a first polynucleotide comprising (i) the nucleotide sequence of SEQ ID NO:1, or (ii) a nucleotide sequence that encodes the amino acid sequence of SEQ ID NO:2; and (b) a second polynucleotide comprising (i) the nucleotide sequence of SEQ ID NO:3, or (ii) a nucleotide sequence that encodes the amino acid sequence of SEQ ID NO:4, wherein the CAG promoter comprises a cytomegalovirus enhancer and beta-actin promoter.

46. The method of claim 44, wherein the first polynucleotide further comprises a woodchuck hepatitis B virus post-transcriptional regulatory element.

47. The method of claim 44, wherein the second polynucleotide further comprises a woodchuck hepatitis B virus post-transcriptional regulatory element.

48. The method of claim 44, wherein said cancer is liver cancer.

49. The method of claim 48, wherein said liver cancer is metastatic.

50. The method of claim 44, wherein the nucleic acid molecule is administered via a portal vein.

51. The method of claim 44, wherein the nucleic acid molecule is administered by muscular injection.
